(19) United States
(12) Patent Application Publication
(10) Pub. No.: US 2006/0101532 A1
(43) Pub. Date: May 11, 2006

(54) Methods for the identification of compounds for the treatment of Alzheimer's disease

(76) Inventors: Ralph A. Nixon, Tarrytown, NY (US); Anne M. Cataldo, Nanuet, NY (US); Paul M. Mathews, Irvington, NY (US)

Correspondence Address: CLARK & ELBING LLP, 101 FEDERAL STREET, BOSTON, MA, 02110, US

(21) Appl. No.: 10/983,422

(22) Filed: Nov. 8, 2004

Related U.S. Application Data
(63) Continuation-in-part of application No. 09/560,124, filed on Apr. 28, 2000, now Pat. No. 6,838,592.
(60) Provisional application No. 60/131,890, filed on Apr. 30, 1999.
(60) Provisional application No. 60/131,991, filed on Apr. 30, 1999.
(60) Provisional application No. 60/140,643, filed on Jun. 23, 1999.
(60) Provisional application No. 60/140,644, filed on Jun. 23, 1999.

(51) Int. Cl. A01K 67/027

(52) U.S. Cl. 800/12; 800/18

(57) ABSTRACT

The invention features methods for identifying compounds useful for the treatment of Alzheimer's disease. The invention also features methods of identifying genes involved in Alzheimer's disease pathology.

### CROSS-REFERENCE TO RELATED APPLICATIONS

This application is a continuation in part of U.S. Utility application Ser. No. 09/560,124 (filed Apr. 28, 2000), which claims benefit from U.S. Provisional Application Nos. 60/131,890 (filed Apr. 30, 1999), 60/131,991 (filed Apr. 30, 1999), 60/140,643 (filed Jun. 23, 1999), and 60/140,644 (filed Jun. 23, 1999), each hereby incorporated by reference.

### STATEMENT AS TO FEDERALLY SPONSORED RESEARCH

The invention was supported in part by National Institutes of Health grant AF 10916. The government has certain rights to the invention.

### BACKGROUND OF THE INVENTION

The invention relates to methods for identifying compounds useful in the treatment of Alzheimer's disease (AD).

Abnormalities of the neuronal endocytic pathway (EP) are the earliest cellular manifestation of sporadic AD (SAD) yet demonstrated. Importantly, EP abnormalities distinguish SAD from certain other subtypes of AD caused by presenilin mutations.

Early endosomes are the site of internalization and initial processing of amyloid precursor protein (APP) and apolipoprotein E (ApoE). They are also a major site of both amyloid β peptide (Aβ) formation and mediate the cellular uptake of Aβ and soluble APP. Individual neuronal endosomes can be as much as 32-fold larger in volume than the normal average and the total endosome volume can be 3-fold higher in SAD brain. Because of a concurrent activation of the lysosomal system (LS), lysosomal acid hydrolases (including proteases) are targetted to early endosomes in increased amounts. These findings, coupled with evidence for the increased mobilization of regulatory proteins of endocytosis, imply a strong upregulation of the EP in Alzheimer's disease.

Greater than 90% of all AD cases are sporadic. Currently there is no suitable animal model for sporadic AD. The early appearance of EP abnormalities and the specificity of these abnormalities in SAD patients suggest that these abnormalities reflect an important pathologic mechanism relevant to the most common form of AD, particularly the β-amyloidogenesis in SAD, the mechanism of which is currently unknown. Thus, there is a need for cellular or animal models of this increased activity, both for increasing the understanding of the pathophysiology underlying AD, and for use in assaying drugs for their use in treating or preventing AD.

Despite the extensive study of AD, the pathogenetic significance of the upregulation of the EP remains unclear. Thus, there is also a long-sought need to understand the importance of increased endocytosis and lysosomal activity in AD.

The clinical entity of human trisomy 21 (Ts21) is the most frequent genetic cause of mental retardation with a prevalence of approximately 1 in 700-800 live births. The phenotype is presumed to be caused by overexpression of a proportion of >300 genes encoded by HSA21. In addition to abnormal central nervous system function during development and adult life, individuals with Ts21 develop AD pathology, including early endosomal abnormalities. A variety of mouse models have been engineered to recapitulate the disease process. An initial murine model of Ts21 is the trisomy 16 (Ts16) mouse. Mouse chromosome 16 (MMU16) has the greatest homology to HSA21, and fetal Ts16 mice display a phenotype similar to Ts21. The Ts16 mouse is limited in that MMU16 is larger than HSA21, and contains genes located on other human chromosomes. Importantly, Ts16 mice die at birth, precluding studies throughout the lifespan. In light of these limitations, criteria for appropriate Ts21 mouse models include three copies of genetic material present on HSA21 that confers the Ts21 phenotype, but not extraneous genetic material outside this region (e.g., the ‘DS’ region). The DS region appears to reside within the q22 region of HSA21. Thus, partial or segmental trisomy of the long arm of HSA21 is an ideal construct for a Ts21 mouse model. In addition, Ts21 mouse models need to survive into adulthood.

Ts65Dn, a segmentally trisomic Ts21 mouse model, fulfills the aforementioned criteria. Ts65Dn are trisomic for a segment of MMU16 orthologous to HSA2 1. Specifically, the distal end of MMU16 is translocated to <10% of the centromeric end of MMU17, creating a small translocation chromosome. This segmental region includes genomic information proximal to App extending to Mx1. Importantly, these mice survive into adulthood.

Ts65Dn have a phenotype that is characteristic of human Ts21. Behavioral studies demonstrate that Ts65Dn mice have learning and memory deficits on a myriad of tasks. Ts65Dn mice also display hyperactivity and neurochemical perturbations. In addition, morphological studies demonstrate abnormalities in specific brain regions of Ts65Dn mice; degeneration of cholinergic basal forebrain (CBF) neurons, volume reduction in the hippocampus and cerebellum, synapse loss, and deficits in synaptic plasticity have been reported.

### SUMMARY OF THE INVENTION

In human SAD and Down syndrome (DS) brains, the total endosomal volume per neuron is 3-fold larger, on average, than in normal control values. Enlarged endosomes are seen in some cortical neurons in infants and third trimester fetuses with DS decades before extracellular β-amyloid deposition is detectable. Similarly, in early stage SAD, endocytic alterations are the earliest cellular change observed in neurons and are present before clinical symptoms are evident and before substantial β-amyloid is deposited.

Our finding of intracellular Aβ within enlarged endosomes in Down syndrome (DS) as early as late gestation suggests a strong association between EP activation and β-amyloidogenesis. Several additional lines of evidence also support the importance of the EP in the production of both forms of Aβ1, Aβ1-40 and Aβ1-42: (i) cultured cells stably transfected with wild type APP, APPswe (APP Swedish mutation or APP670/671), or APPswe/V717L (Indiana mutation), combined with deletion of the APP C-terminal endocytic targeting signal, showed a decrease in Aβ1-40 secretion and a decrease in the ratio of Aβ42/40, suggesting a decrease in Aβ42 production (Stokin et al., 6th Int'l. Conf. on Alzheimer's disease, Amsterdam, 1998); (ii) the expression of the dominant negative dynamin mutant, which prevents endocytosis, showed decreased ratios of secreted Aβ1-42/Aβ1-40 for the three foregoing APP-expressing cell lines (Stokin et al., 6th Intl. Conf. on Alzheimer's disease, Amsterdam, 1998); and (iii) cell models using chimeric forms of APP that were retained in the ER, were directed to the lysosome, or to the cell surface showed that Aβ1-40 was produced mainly in the EP, and that Aβ1-42 was produced both in the endoplasmic reticulum/cis-Golgi apparatus and in the EP (Soriano et al., 6th Intl. Conf. on Alzheimer's disease, Amsterdam, 1998; Soriano et al., J. Biol. Chem. 274:32295-32300, 1999).

We hypothesize that the endocytic pathway abnormalities that alter APP processing and lead to the increased production of Aβ in SAD and DS are accentuated by the APOE ε4 allele, implying mechanistic links among EP alterations, β-amyloidogenesis, and genetic susceptibility for AD. Activation of neuronal endocytosis is not a consequence of Aβ overproduction or β-amyloid deposition, as we have not detected EP abnormalities in individuals with FAD caused by PS mutations that exhibit abundant P-amyloid deposition. Using compartment-specific markers to identify early endosomes combined with markers of endosomal fusion and recycling, we have shown an upregulation of both endosomal fusion and recycling, indicating increased EP activation.

Accordingly, the invention features methods for identifying compounds for the treatment of AD.

In a first aspect, the invention features a method for identifying a candidate compound as a compound that is useful for the treatment of AD that includes: a) providing a Tn65Dn mouse; b) administering the candidate compound to the mouse; and c) measuring the abnormal activity of the EP and/or a change in Aβ generation. A decrease in the EP activity compared to the activity in a control Tn65Dn mouse not contacted with the candidate compound identifies the candidate compound as a compound that is useful for the treatment of AD. Similarly, a decrease in AP generation in a mouse, relative to a mouse not contacted with the candidate compound, also identifies a compound as one that is useful for the treatment of AD.

In a second aspect, the invention features a method for identifying a candidate compound as a compound that is useful for the treatment of AD. The method includes: a) providing a cell from a Tn65Dn mouse or a fibroblast from a patient with trisomy 21; b) contacting the cell with the candidate compound; and c) measuring the abnormal activity of the EP, wherein a decrease in the EP activity, compared to the activity in a control cell (also from a Tn65Dn mouse) not contacted with the candidate compound, identifies the candidate compound as a compound that is useful for the treatment of AD. As described above, Aβ generation can also be used as a measurement; a decrease in Aβ generation in a cell mouse, relative to the control cell, also identifies a compound as one that is useful for the treatment of AD. In preferred embodiments, the cell is from a cell line derived from the Ts21 patient or a Ts16 mouse (e.g., a Ts65Dn mouse). A preferred cell line includes a fibroblast cell line, a neuronal cell line, or a neuroblastoma cell line. In related embodiments, the cell is a fibroblast, a neuron, or an endothelial cell.

In a third aspect, the invention features a method for identifying a candidate compound as a compound that is useful for the treatment of AD. The method includes: a) providing a cell expressing a recombinant nucleic acid that causes abnormal activity of the endocytic pathway; b) contacting the cell with the candidate compound; and c) measuring the activity of the EP or Aβ generation activity, wherein a decrease in the abnormal activity, relative to the activity in a control cell expressing the recombinant nucleic acid but not contacted with the candidate compound, identifies the candidate compound as a compound that is useful for the treatment of AD.

In preferred embodiments, the recombinant nucleic acid includes rab5, 46 kDa mannose 6-phosphate receptor (MPR46), cathespin (Cat D), or nucleic acids encoding other lysosomal hydrolases trafficked by the mannose 6-phosphate tag. In various embodiments, the cell is from a fibroblast cell line, a neuronal cell line, or a neuroblastoma cell line or is a fibroblast, a neuron, or an endothelial cell.

In preferred embodiments of the second and third aspects, the cell is in an animal, or the cell is in vitro.

In a fourth aspect, the invention features a method for identifying a candidate compound as a compound that is useful for the treatment of AD. The method includes a) providing a mouse expressing a transgene that results in increased activity of the EP or using the Tn65Dn mouse (or a derivative thereof that harbors only the genes on MMU16 required for the phenotype); b) administering the candidate compound to the mouse; and c) measuring the activity of the EP or Aβ formation activity, wherein a change in the abnormal activity, relative to the activity in a mouse expressing the transgene but not contacted with the candidate compound, identifies the candidate compound as a compound that is useful for the treatment of AD.

In a preferred embodiment, the recombinant nucleic acid includes rab5, MPR46, Cat D, or other lysosomal hydrolases trafficked by the mannose 6-phosphate tag.

In preferred embodiments of the first, second, third, and fourth aspects, the abnormal activity includes increased endosomal fusion, increased endosomal recycling, upregulation of MPR46, accumulation of lysosomal hydrolases in early endosomes, or accumulation of Aβ in early endosomes, or failure of the maturation of early endosomes through later steps of endocytosis.

In a fifth aspect, the invention features a method for identifying a candidate compound as a compound that is useful for the treatment of AD. This method includes the steps of: (a) providing a non-human animal; (b) administering to the animal a compound that induces endosomal-lysosomal dysfunction; (c) administering to the animal a candidate compound; and (d) measuring neurodegeneration in the animal. A decrease in neurodegeneration, relative to an animal administered the compound that induces endosomal-lysosomal dysfunction but not the candidate compound, identifies the candidate compound as a compound that is useful for the treatment of Alzheimer's. Preferably, the animal is a Tn65Dn mouse or a mouse expressing a transgene comprising a recombinant nucleic acid that increases activity of the endocytic pathway (such as the ones described lo herein). In various embodiments, the compound that induces lysosomal dysfunction is a lysosomal protease inhibitor, the animal has increased endocytic pathway activity relative to a normal animal.

In another aspect, the invention features a method of identifying a gene as a gene involved in Alzheimer's disease pathology. This method includes the steps of: (a) providing a cell from a Tn65Dn mouse or a cell from a human with trisomy 21; (b) contacting the cell with an agent that reduces the expression of a gene that is present in the cell in three copies; and (c) measuring the activity of the endocytic pathway. A decrease in this activity, compared to the activity in a control cell not contacted with the agent, identifies the gene as a gene that a gene involved in Alzheimer's disease pathology.

The activity of the endosomal pathway may be, for example, endosomal fusion, endosomal recycling, expression of MPR46, accumulation of lysosomal hydrolases in early endosomes, or accumulation of Aβ in early endosomes.

The cell may be in vitro or in vivo. The cell may be from a cell line derived from a mouse or a human (e.g., a fibroblast cell line, a neuronal cell line, or a neuroblastoma cell line).

In one embodiment, the gene is selected from the group consisting of Adamts1, Adamts5, Ankrd3, Atp5a, Atp5o, B3galt5, Bace2, Bach1, C21orf108, C21orf18, C21orf25, C21orf4, C21orf5, C21orf51, C21orf59, C21orf6,C21ord63, C21orf66, C21orf7, Cbr1, Cbr3, Cct8, Chaf1b, Cldn17, Cldn8, Clic6, Cryzl1, Ctdbp, Donson, Dscam, Dscr1, Dscr2, Dscr3, Dscr5, Dyrk1a, Erg, Ets2, Fam3b, Gabpa, Gart, Grik1, Hlcs, Hmgnl, Hunk, Ifnar1, Ifnar2, Ifngr2, Igsf5, Il10rb, Itsn1, Jam2, Kcne1, Kcne2, Kcnj15, Kcnj6, Kiaa0136, Kiaa0184, Krtap11-1, Mrpl39, Mrps6, Mx1, Mx2, Olig2, Pcbp3, Pcnt2, Pcp4, Pdxk, Pjkl, Psmd4, Runx1, Sh3bgr, Sim2, Slc5a3, Sod1, Son, Synj1, Tiam1, Tmprss2, Ttc3, Usp16, Wdr9, Wrb, Znf294, and Znf295.

By “abnormal activity of the endocytic pathway” is meant an activity that is normally not observed in a control cell or animal. Abnormal activities of the EP include: (i) increased endocytic rates; (ii) increased endosomal fusion and recycling; enlargement of endosomes (iii) accumulation of lysosomal hydrolases in early endosomes; (iv) increased activity of lysosomal hydrolase in early endosomes; (v) upregulation of MPR46; and (vi) accumulation of β-amyloidogenic fragments in early endosomes, or impaired endosomal proteolysis or altered trafficking of early endosomes or its constituents to later compartments of the endocytic pathway.

A compound that normalizes the abnormal activity of the endocytic pathway is one that exhibits any diminution in one or more of the foregoing activities. The decrease in abnormal activity is preferably by at least 5%, more preferably by at least 10%, and most preferably by at least 25%, 50% or more. The percent change is usually measured for a period of hours or days, but can be measure in terms of weeks or even longer.

By “endosomal-lysosomal dysfunction” is meant an alteration in one function of the lysosomal system. The alteration is preferably by at least 5%, more preferably by at least 10%, and most preferably by at least 25%, 50% or more.

The invention provides methods for identifying drugs useful for the treatment or prevention of AD. Additionally, the invention provides new drug targets for rational drug design.

Other features and advantages of the invention will be apparent from the following description of the preferred embodiments thereof and from the claims.

### BRIEF DESCRIPTION OF THE DRAWINGS

FIGS. 1A-1C are a series of photographs of tissue sections of prefrontal cortex of a control (FIG. 1A) and two early stage AD cases (FIGS. 1B and 1C) labeled with anti-rabS antibody.

FIGS. 1D and 1E are photographs of adjacent tissue sections of prefrontal cortex from an early stage AD case processed for Aβ immunohistochemistry (FIG. 1D) or stained by Bielschowsky stain (FIG. 1E).

FIG. 1F is a graph showing that the average endosomal volume per neuron in the early stage AD cases was elevated compared to that of normal controls (control mean, 1.88%; possible AD mean, 3.68%; SAD mean, 5.04%).

FIGS. 2A-2E are a series of photographs of tissue sections of prefrontal cortices from a fetus (FIG. 2A), infant (FIG. 2B-2D), or young child (FIG. 2E) with DS immunolabeled for rabS.

FIGS. 3A and 3B are a series of photographs of tissue sections of the prefrontal cortex from early stage AD brains carrying the APOE ε4 genotype immunolabeled for rab5.

FIG. 3C is a schematic illustration showing that the endosomal volume per neuron was 50% higher in individuals carrying one or both copies of APOE ε4.

FIGS. 4A-4E are a series of photographs showing early endosomal abnormalities in the cerebral vascular epithelium in heritable Dutch CAA (FIGS. 4A-4C) and late onset FAD carrying the APOE ε4 allele (FIG. 4B).

FIGS. 5A-5D are a series of photographs of immunofluorescence labeling of human MPR46 in control murine L cells (FIG. 5A), or L cells stably-transfected with human MPR46 (FIG. 5B), MPR-HA (FIG. 5C), or MPR-HAY (FIG. 5D).

FIGS. 5E and 5F are photographs of control L cells (FIG. 5E) or L cells stably-transfected with MPR-HAY (FIG. 5F) showing that MPR-HAY-transfected cells preferentially took up BODIPY-pepstatin from the medium.

FIG. 5G and 5H are photographs of immunofluorescence labeling of human MPR in N2a cells expressing MPR46 (FIG. 5G) or MPR-HAY (FIG. 5H).

FIG. 5I is a schematic illustration showing overexpression of MPR46 or a MPR46 trafficking mutant results in increased Aβ levels in the culture medium in cells overexpressing human APP.

FIG. 5J is a schematic illustration showing overexpression of MPR46 trafficking mutant results in increased Aβ levels in the culture media of cells that are not overexpressing APP.

FIG. 6A is a photograph of a western blot showing rab5 immunoreactivity in control L cells and L cells stably-transfected with rab5.

FIG. 6B is a photograph of L cells stably-transfected with rab5Q₇₉L showing enlarged endosomes (arrow).

FIGS. 6C and 6D are photographs of control L cells (FIG. 6C) or L cells overexpressing wild-type rab5 (FIG. 6D) showing increased internalization of Cy3-labeled transferrin.

FIG. 7A is a photograph of a DNA agarose gel identifying 2 of 11 pups within a litter as carrying the Thy1.1: MPR46 transgene.

FIG. 7B is a schematic illustration showing increased Aβ levels in the brain of a transgenic mouse expressing human MPR46 under the Thy1.1 promoter.

FIGS. 8A and 8B are photographs showing rab5 immunoreactivity in cortical neurons from control mice (FIG. 8A) and mice with segmental trisomy 16 (Ts65Dn; FIG. 8B).

FIGS. 8C-8F are photographs showing rab5 immunoreactivity in cortical neurons, particularly showing more labeling in Ts65Dn mice (FIG. 8F) compared to control mice (FIG. 8E).

FIGS. 9A-C are photographs showing intracellular Aβ only in neurons of Ts65 on mice using antibody 4G8 (FIG. 9B) and an Aβ40 specific antibody (FIG. 9C) but not in neurons of a control mouse (FIG. 9A).

FIGS. 9D-F are photographs of immunofluorescence labeling showing the coincidence (FIG. 9F) of rab5 (FIG. 9D) and AP (FIG. 9E) in Ts65Dn neurons.

FIG. 10 is a schematic illustration showing increased levels of AP in the brains of Ts65Dn mice vs. control mice.

FIGS. 11A and 11B are photographs showing lipotuscin autofluorescence in sections of leupeptin-treated but not vehicle-treated brains.

FIGS. 11C and 11D are photographs showing increased Cat D immunolabeling in sections of leupeptin-treated vs. vehicle-treated brains.

FIG. 11E is a photograph of a western blot showing increased Cat D protein levels in the brain of leupeptin-treated mice when compared to control mice.

FIG. 11F is a schematic illustration showing increased Cat D activity in the brains of leupeptin-treated mice when compared to control mice. FIG. 12 is a photograph of a western blot showing an increase in the lower molecular weight forms of tau in leupeptin-treated mice.

FIGS. 13A and 13B are photographs of Cat D immunoreactivity in the brains of normal mice infused with vehicle (FIG. 13A) or leupeptin (FIG. 13B).

FIGS. 13C and 13D are photographs of Cat D immunoreactivity in the brains of Ts65Dn mice infused with vehicle (FIG. 13C) or leupeptin (FIG. 13D).

FIGS. 14A-14D are photographs of Niss1 staining of brain sections of a non-transgenic mouse infused for 4 weeks with leupeptin (FIGS. 14A and 14C) and of a PS_{M146}L/APP_{swe} transgenic mouse infused for 4 weeks with leupeptin (FIGS. 14B and 14D).

FIGS. 15A-15C are photomicrographs at low modification showing Niss1 staining the extent of the cortical mantle in a non-transgenic mouse infused with vehicle (FIG. 15A), a non-transgenic mouse infused with leupeptin (FIG. 15B), and a PS_{M146L}/APP_{swe} transgenic mouse infused with leupeptin (FIG. 15C).

### DETAILED DESCRIPTION OF THE INVENTION

We have discovered that early endosomal abnormalities in SAD precede significant β-amyloid deposition and neurofibrillary pathology and that it is very likely that these abnormalities lead to the resulting neuropathology in at least SAD patients. We have further discovered that these endosomal abnormalities can be mimicked in cell or animal models by either increasing endocytosis or by altering trafficking in the EP in transgenic mice or transformed cell lines. Finally, we have found that the segmental trisomy 16 mouse, Ts65Dn, previously discounted as a suitable mouse model of AD, displays the early hallmarks of endosomal abnormalities.

Each of the transgenic mice and transformed cell lines serve as models for understanding the cell biology underlying AD, as well as for testing candidate drugs for their efficacy and safety in the treatment of this prevalent disease.

### EXAMPLE 1

### Endosomal Abnormalities Precede β-amyloid Deposition and Early Neurofibrillary Change

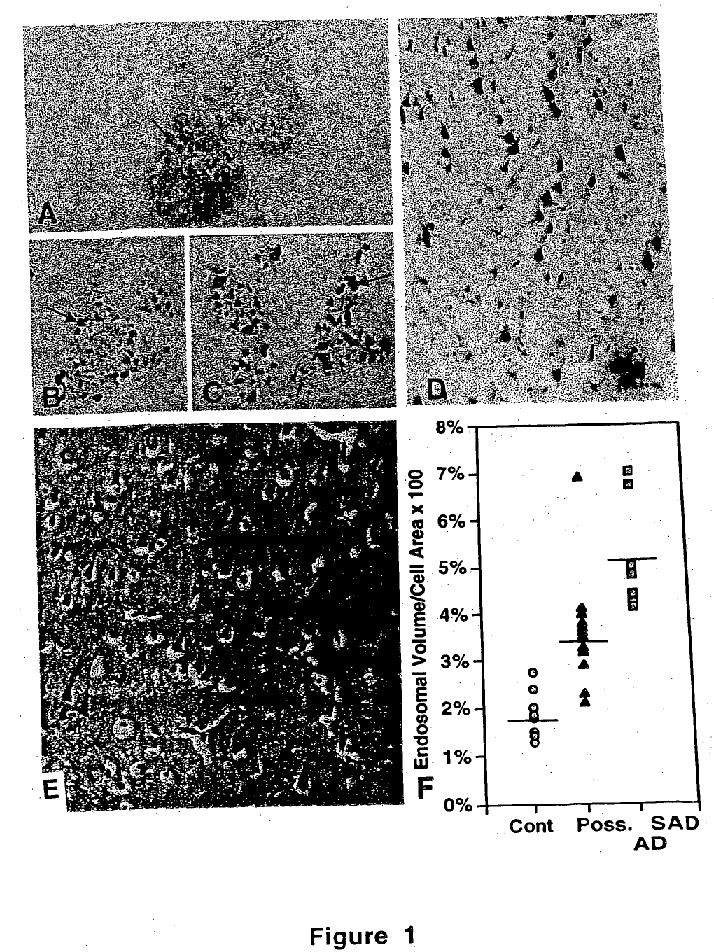

We studied SAD cases that met the CERAD criteria of “possible AD,” with neuropathology limited to the entorhinal cortex/hippocampus. Abnormally large endosomes were prominent not only in neurons of the entorhinal cortex, and CA2 and CA3 of the hippocampus, but also in laminae III and V of the prefrontal cortex (FIG. 1), where deposits of β-amyloid were scarce and neurofibrillary pathology was negligible by immunostaining for early tau abnormalities. These results suggested that endosomal abnormalities are the earliest cellular disturbance yet to be identified in possible AD, preceding all but the first traces of β-amyloid deposition. Studies of DS suggest that early endosomal changes can be demonstrated even earlier when individuals can be identified who are destined to develop AD. These changes could be identified before any trace of β-amyloid was present.

Abnormal activity of the EP distinguish SAD and DS from FAD caused by PS mutations. Pyramidal neurons in individuals with moderate to severe AD caused by PS1 mutations do not display the early endosomal abnormalities seen in SAD, late onset FAD-APOE ε4, DS, or APP 717 mutations. Because PS-FAD is associated with high levels of β-amyloid deposition, these results suggest that endosomal alterations are not a consequence of β-amyloid accumulation. That PS mutations do not alter endosomal function is consistent with the hypothesis that AD caused by PS1 mutations may occur via secretory pathway organelles (e.g., endoplasmic reticulum and the Golgi apparatus) rather than the EP. In contrast to EP alterations, LS activation is accentuated in PS-FAD, including in neuronal populations that are less vulnerable in SAD (e.g., laminae II and IV of prefrontal cortex).

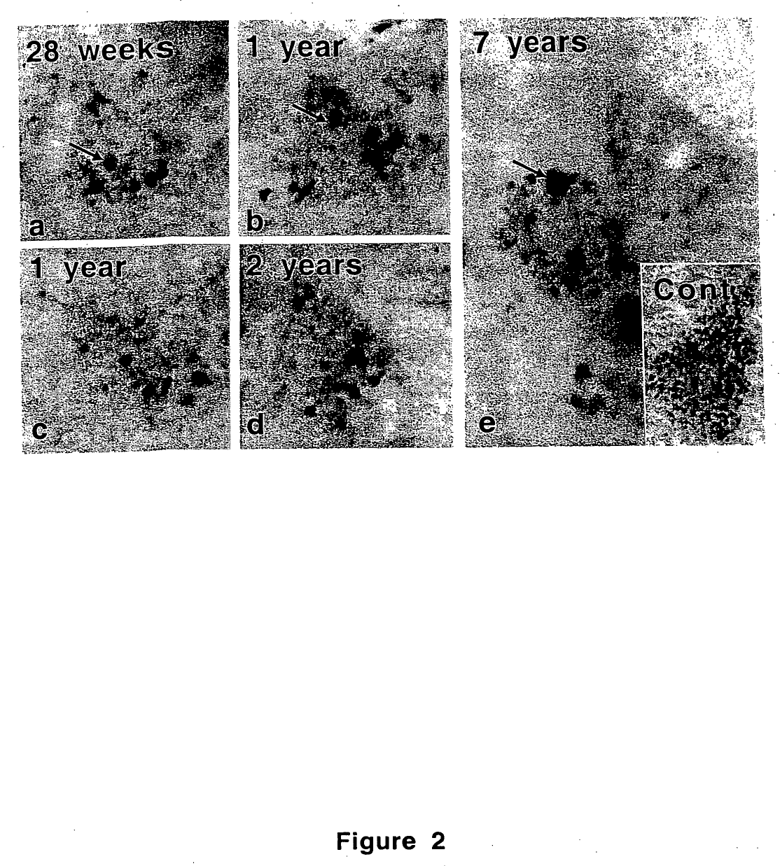

Most individuals with DS develop neuropathological features indistinguishable from SAD at an early age. Using specific probes for early endosomes and proteins involved in the regulation of endosomal fusion (rab5, rabaptin 5, and EEA 1), we found that, like SAD, neurons from adult DS brains exhibited abnormally large early endosomes. Similarly, in tissue obtained from seven cases of fetal, infant and young DS brains, ranging in age from 28 weeks gestation to 12 years, we observed that swollen endosomal profiles greater than 1 um³ and similar to those seen in adult DS were present in many pyramidal neurons (FIG. 2). No detectable β-amyloid or abnormal neurofibrillary changes were seen in the brain parenchyma of the young DS brains. We observed that intraneuronal AP colocalized with enlarged endosomes of infant DS less than 1 year of age, consistent with the observations of Teller et al. (Nature Med. 2: 93-95, 1996), who showed increased Aβ1-42 levels in fetal DS brains as early as 21 weeks gestation.

Given that endosomes are potentially a major site of Aβ production, the presence of atypically large early endosomal profiles suggests that Aβ production in early endosomes may be accelerated in SAD long before it accumulates extracellularly. If so, other factors such as alterations in Aβ clearance are likely to be needed to promote β-amyloid deposition.

In the above studies of early stage AD, we show morphologic and biochemical alterations of the early endocytic pathway indicative of increased endocytosis, early endosomal fusion and recycling using compartmental-specific markers rab5, EEA1, rabaptin 5, and rab4. Because early endosomes are in direct contact with late endosomes and transport material to the late endosomes prior to subsequent degradation in lysosomes, we extended these analyses by examining later steps in the endocytic pathway using antibodies to rab7, which regulates the delivery of early endosomal constituents to late endosomes and late endosomal fusion, and lysobisphosphatidic acid (LBPA), a slowly degraded phospholipid restricted to late endosomal membranes. We studied a series of sporadic AD cases that met the CERAD criteria for “possible AD” with neuropathology limited to the transentorhinal cortex. Abnormally large late endosomes were seen using both rab7 and LBPA antibodies in neurons of the temporal and prefrontal cortices and hippocampus. The enlarged rab7-positive late endosomal vesicles colocalized with most LBPA-positive vesicles. In neurons, these vesicles accumulated in the perinuclear region of the cell much like after rab7 overexpression and following treatment with cholesterol. Double-label immunofluorescence showed restricted overlap of enlarged rab7- and LBPA-positive vesicles with EEAL and no overlap with rab4. Neurons in all regions examined in control brains showed no evidence of late endosomal enlargement with rab7 or LBPA antibodies. Because late endosomes play a central role in protein and vesicular sorting, mannose 6-phosphate receptor and lysosomal hydrolase transport, protein degradation, and are the single site where the early endosomal, autophagic, and lysosomal systems all intersect, dysfunction of the late endosome is likely have important pathological implications in AD.

### EXAMPLE 2

### Accentuation of Early Endosomal Abnormalities in Individuals Carrying the APOEε4 Allele

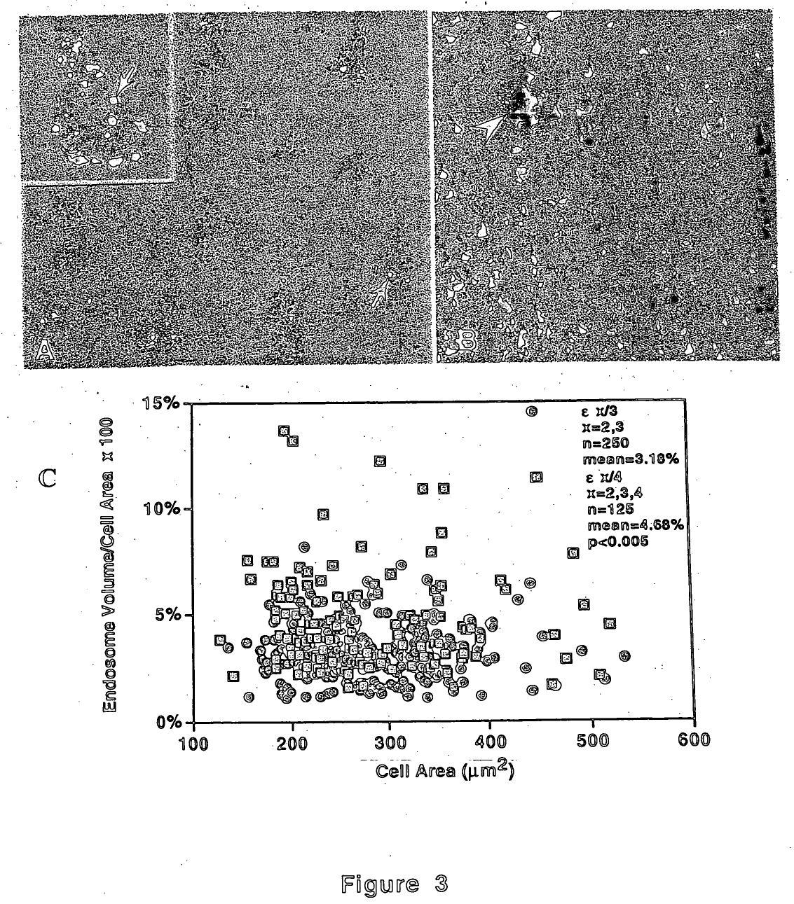

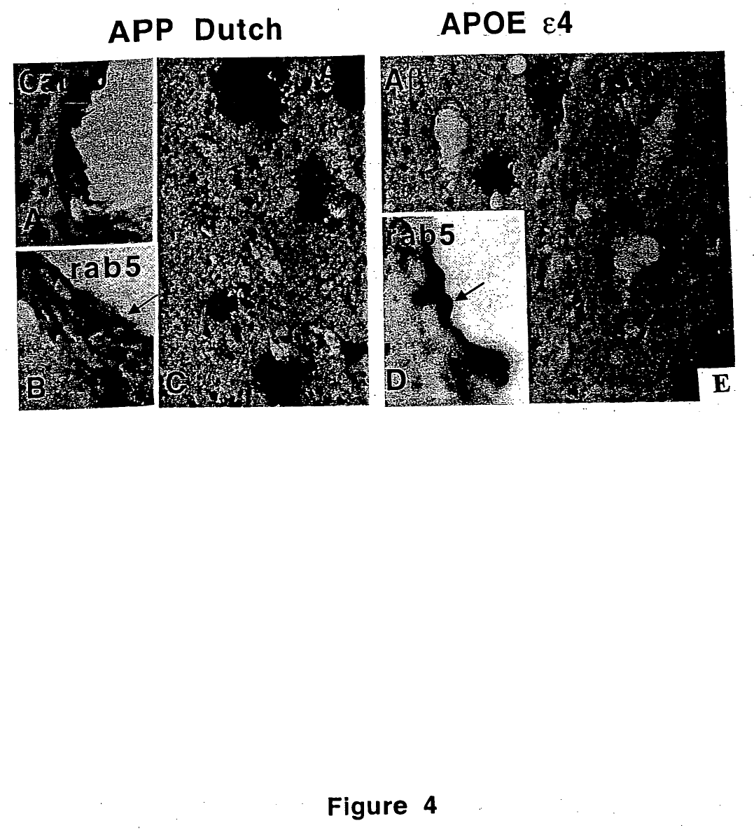

We examined tissue sections from a total of 15 early stage cases, consisting of 10 cases with APOE ε3 or APOE ε2 alleles and 5 cases carrying one or both copies of the APOE ε4 allele. These brains exhibited minimal histopathology and met the CERAD criteria of “possible AD.” Neocortical sections were immunostained with rab5 antiserum and morphometric analyses were performed on groups of 25 neurons per brain. We found that the average mean endosomal volume/neuron in the early stage cases with the ε4 alleles was 50% larger than the AD cases with the ε3 or ε2 alleles (FIG. 3), suggesting a link between EP alterations and inheritance of the APOE genotype. Altered endocytosis would be expected to influence ApoE internalization and function, a prediction consistent with the observation of elevated ApoE immunoreactivity in pyramidal neurons in AD.

### EXAMPLE 3

### Transfected Cell Models of Endosomal and Hydrolase Trafficking Abnormalities in AD Brain

We produced fibroblast cell lines that have been genetically engineered to model the enlargement of early endosomes, increased endocytosis, and increased trafficking of proteases to early endosomes that we have observed in AD brain. We believe that this strategy of mimicking pathological changes in the EP known to occur in AD brain has advantages over currently used manipulations of APP trafficking that may not be representative of disease-relevant mechanisms

MPR46

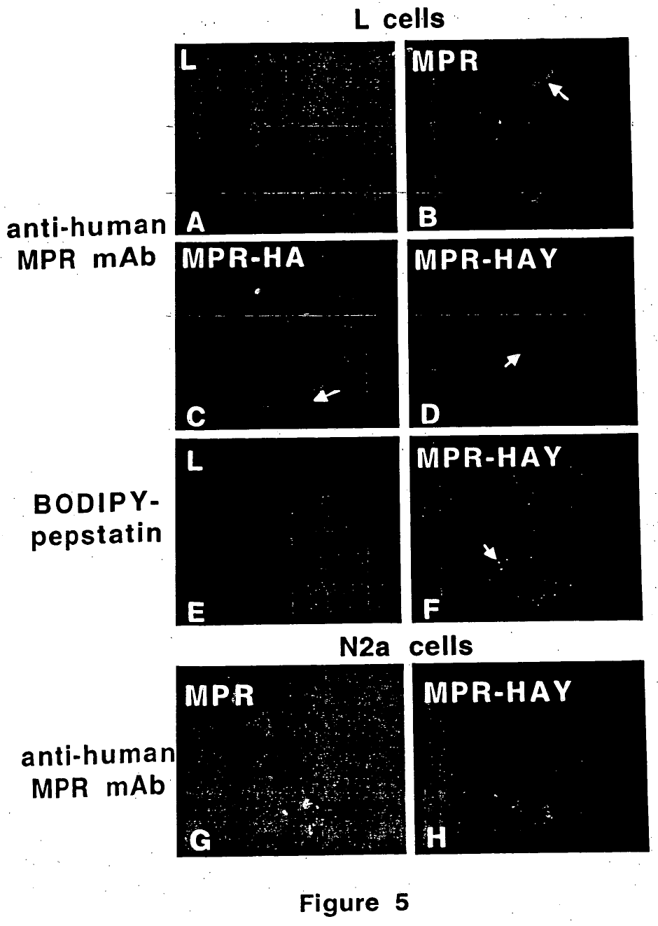

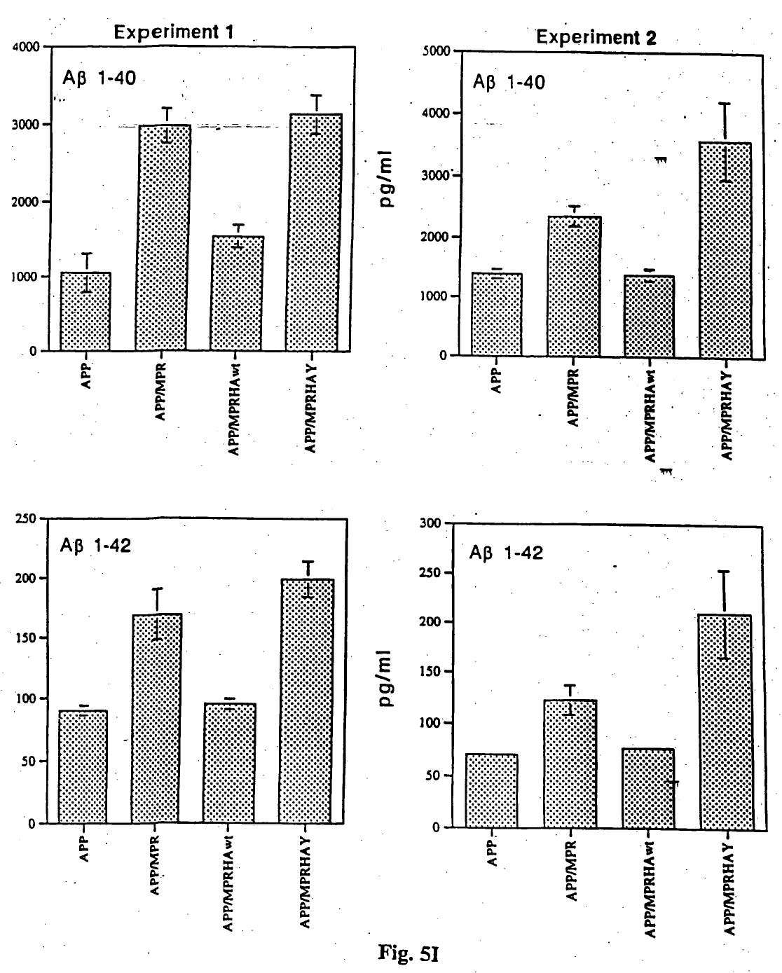

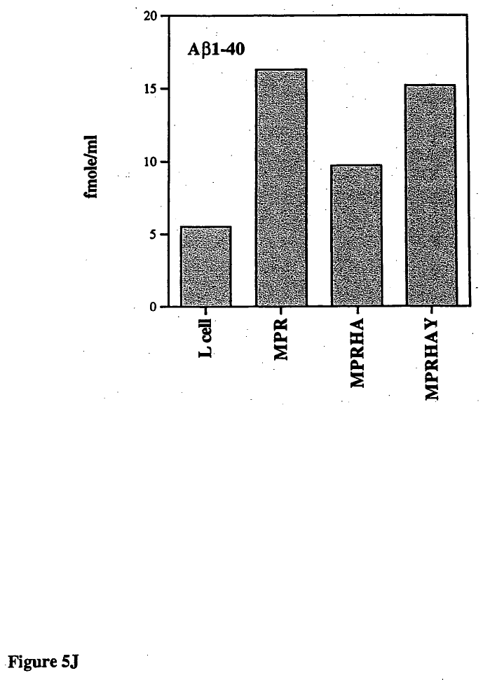

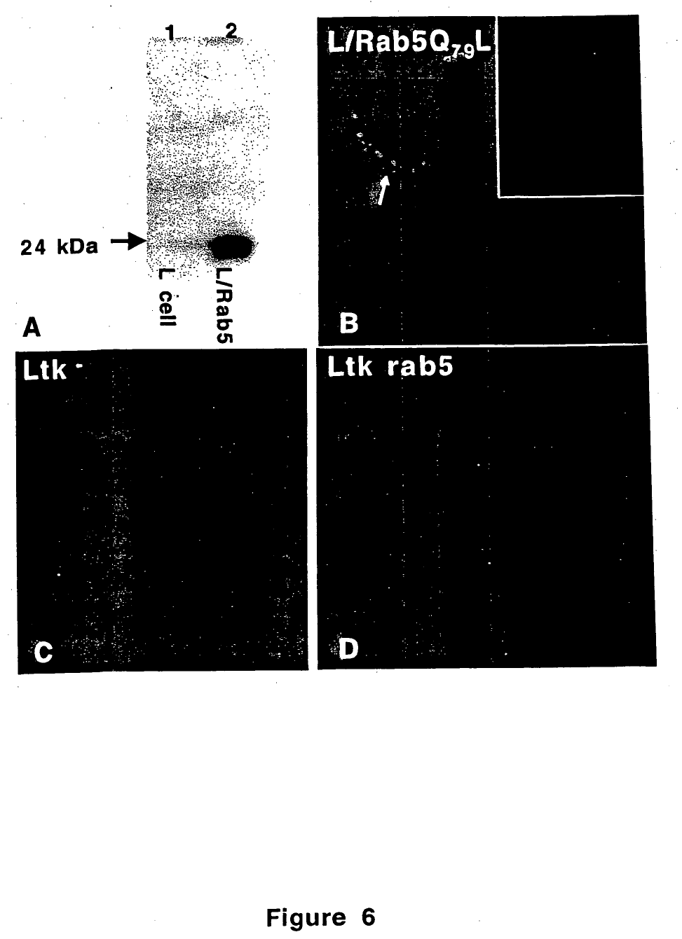

To mimic the hydrolase mistrafficking seen in AD, we isolated a cDNA encoding the human MPR46 and expressed this in stably transfected murine fibroblast-like cell lines (L cell; FIG. 5A-5J). In addition, we constructed two targeting mutants of MPR46 by replacing its C-terminal tail with that of the plasma membrane influenza virus hemagglutinin (MPR-HA) or a modified HA tail containing a tyrosine-residue endocytosis motif (MPR-HAY; Roth et al., J. Cell Biol. 102:1271-1283). The wild-type MPR46 predominantly localized to perinuclear vesicles consistent with the trans-Golgi sacs and late endosomes. As predicted, MPR-HA was expressed at the cell surface, while MPR-HAY was localized primarily to small vesicles, which we have shown to be early endosomes by both rab5 immunolabeling and transferrin uptake. Using a fluorescent-tagged pepstatin, an inhibitor of Cat D that binds to the proteolytically active enzyme, we demonstrated that expression of the MPR-HAY chimera partially distributes this lysosomal hydrolase to early endosomes. Overexpression of wild-type MPR46 appears to similarly redistribute Cat D to early endosomes, although to a lesser extent. Thus, by expressing MPR-HAY and/or overexpressing MPR46, we can model the increase of active lysosomal hydrolases in early endosomes seen in SAD.

We introduced these constructs into L cells overexpressing wild-type human APP to determine the effect of increased hydrolases in early endosomes on APP processing. A murine L cell line overexpressing APP was transfected with MPR46 (APP/MPR), a plasma membrane-targeted MPR46 (APP/MPRHAwt), or a MPR46 construct that is preferentially trafficked to the early endosome (APP/MPRHAY). Cells were incubated for 8 hours following a media change, and the amount of Aβ1-40 and Aβ1-42 secreted into the medium during this time were determined by ELISA. Overexpression of MPR46 increased Aβ1-40 and Aβ1-42 generation above control (APP) cells (FIG. 5I). As we predicted, expression of the plasma membrane-targeted MPRHAwt construct did not affect Aβ generation. Expression of the early endosome-trafficked MPRHAY, however, increased Aβ1-40 and Aβ1-42 production over control and, in one experiment, beyond the increase seen with MPR46 expression (FIG. 51). We confirmed these results in L cells that were not overexpressing APP. As seen in APP overexpressing L cells, overexpression of MPR46 increased the amount of endogenous mouse Aβ1-40 approximately three times above control cells (FIG. 5J). While in this experiment MPRHAwt had a small effect on Aβ1 -40 production, MPRHAY expression had a much larger effect, similar to MPR46 overexpression (FIG. 5J). It is likely, based on these findings, that the redistribution of lysosomal hydrolases in human AD resulting from increased expression of MPR46 is likely to contribute to increased AP production. Thus, use of MPR46 and MPR46 trafficking mutants to mistraffick hydrolases provides a model for these changes in vitro and in vivo.

rab5

To model the enlargement of early endosomes and increased endocytosis seen in AD, we made stably transfected cell lines overexpressing rab5 (FIG. 5A-5J). Immunolabeling for rab5 showed enlarged endosomes, similar to those seen in neurons from individuals with SAD, when compared to non-transfected cells. In addition, we also constructed a cDNA encoding the GTP-hydrolysis deficient rab5 mutant Q₇₉L (Stenmark et al., EMBO J. 13: 1287-1296, 1994) and expressed this in L cells. Expression of the rab5 Q₇₉L mutant results in increased endocytosis and the fusion of early endosomes into large vacuoles. Confirming that rab5 overexpression led to abnormal activity of the EP, we demonstrated both increased uptake of fluid phase markers (FITC-dextran) and increased receptor mediated endocytosis (transferrin).

The ability of a compound to counteract the effect of rab5 overexpression and rab5Q₇₉L expression on endocytosis is determined, for example, by immunolabeling using antibodies to rab5, EEA 1, and/or rabaptin 5, followed by computerized morphometric analysis. Functional uptake studies can also be used to assess fluid phase and receptor mediated endocytosis. Redistribution of hydrolases, specifically to early endosomes by MPR46 and/or MPR-HAY, are determined with assays such as immunolabeling, BODIPY-pepstatin uptake, or metabolic labeling followed by immunoprecipitation of specific lysosomal hydrolases. Administration of a candidate compound to neuroblastoma cells overexpressing MPR46 or rab5, or expressing the MPR chimeras, can also be assayed by directly evaluating APP processing or Aβ production under conditions of increased endocytic uptake or increased hydrolase delivery to early endosomes.

### EXAMPLE 4

### Transgenic Mice Models Having Endosomal and Hydrolase Trafficking Abnormalities in AD Brain

Transgenic mouse lines overexpressing any of the foregoing constructs provide useful animal models for identifying drugs useful for the treatment of AD. These mice can be made using standard techniques. If desired, the expression of the transgenes can be restricted to neurons by use of a promoter such as the Thy1.1, neuron-specific enolase, or Tα1 α-tubulin promoters. The transgenic mice described herein can be crossed with each other, with a mouse that has increased AD-like pathology, a mouse that is expressing a polypeptide that has a mutation found in a human with AD (e.g., APP, PS-1, or PS-2), or with a reporter mouse. Any of the assays of abnormal activity of the EP can be performed to monitor the effectiveness of a candidate compound in decreasing the abnormal activity of the EP. The candidate compound can be directly administered to the mouse. Alternatively, cells from the mice, such as fibroblasts, endothelial cells, or neurons, can be assayed in vitro.

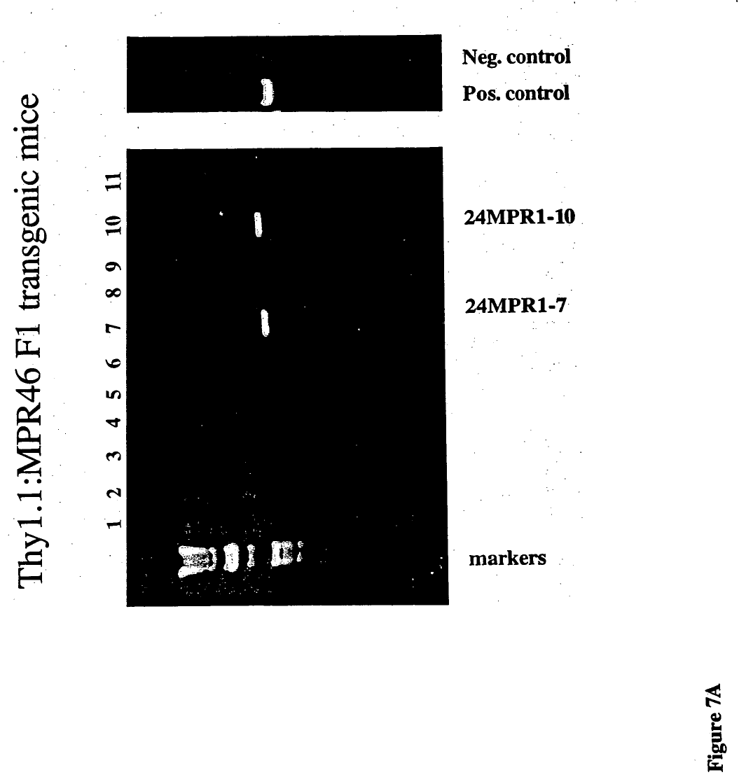

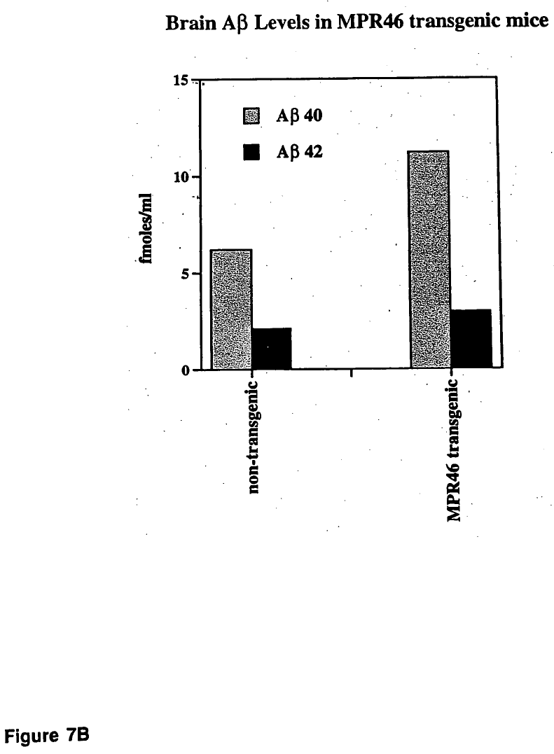

As one example of such a transgenic mouse, we have constructed a mouse overexpressing MPR46 within neurons under the Thy-1.1 promoter. Following DNA microinjection and implantation of approximately 75 single cell embryos, 29 pups were raised to weaning. From these, two mice were identified that contained the Thy-1.1 MPR46 transgene construct. Both of these founders were shown to transmit the Thy-1.1: MPR46 transgene to F1 offspring. FIG. 7A identifies 2 of 11 F1 offspring of one such founder as carrying the Thy-1.1: MPR46 transgene. Brain Aβ levels were compared between one such F1 Thy-1.1: MPR46 transgenic mouse and a non-transgenic litter mate (FIG. 7B). While the increase in brain AP seen in this four week old heterozygous Thy-1.1 MPR46 transgenic mouse was modest (approximately 1.7 times of Aβ1-40), it is likely that an older mouse will show an even greater increase in Aβ. Additionally, a greater effect is likely to be seen in homogozygous transgenic mouse; finally, additional transgenic lines can be generated with higher levels of MPR46 expression.

Thy-1.1: MPR46 Transgenic Mouse Methods

Neuron specific expression of human MPR46 is driven by the Thy-1.1 promoter, which has been used successfully in mice transgenic for mutant APP that show amyloid plaque deposition (Sturchler-Pierrat et al., Proc. Natl. Acad. Sci. USA 94:13287-13292, 1997). The Thy-1.1 promoter is valuable in that high levels of transgene expression have been obtained and that expression is restricted within the CNS to neurons. An 8.2 kb EcoRI mouse genomic fragment containing the whole Thy-1.1 gene forms the core of the transgene constructs (Chang et al., Proc. Natl. Acad. Sci. USA 82:3819-3823, 1985; Gundersen et al., Gene 113:207-214, 1992; Luthi et al., J. Neurosci. 17:4688-4699, 1997). After preparation by standard methods, plasmid was digested with the appropriate restriction enzymes to remove vector sequences, isolated by agarose-gel electrophoresis, and further purified by dialysis in ultra-pure water. Single cell embryos obtained from C57BL6×CBA/2 F1 hybrids were used for injection. Founder transgenic mice were identified and transgene copy number estimated by Southern blot analysis of restriction digested tail DNA using hybridization to a fragment unique to the transgene. Progeny were screened by PCR using human-specific primers. Some F1 progeny from the two transmitting founders were examined at 4 weeks of age.

### EXAMPLE 5

### Fibroblasts From DS Human Patientsexhibit Abnormal Activity of the Endocytic Pathway

Explants of minced skin tissue taken antemortem from the forearm of two donors, aged five years and thirteen months, with the typical features of DS (Trisomy 21; karyotype 47, XY, +21) and from minced umbilical cord tissue of a therapeutically aborted fetus of twenty-two weeks gestation (karyotype 47, XY, +21) were grown to confluency on glass coverslips and fixed for morphologic inspection of early endosomal compartments and localization of the Aβ peptide. In each of the cell lines from the DS cases, we observed intracellular localization of Aβ in compartments close to the cell surface that were consistent with early endosomes. Thus, the abnormal activity of the EP observed in SAD and DS brains are mimicked by cultured fibroblasts from DS individuals. This finding indicates that DS fibroblasts would be a suitable model to screen candidate compounds for their ability to prevent the earliest stages of AD.

### EXAMPLE 6

### Molecular Analysis of Early Endosomal Abnormalities in Ts65Dn Mice

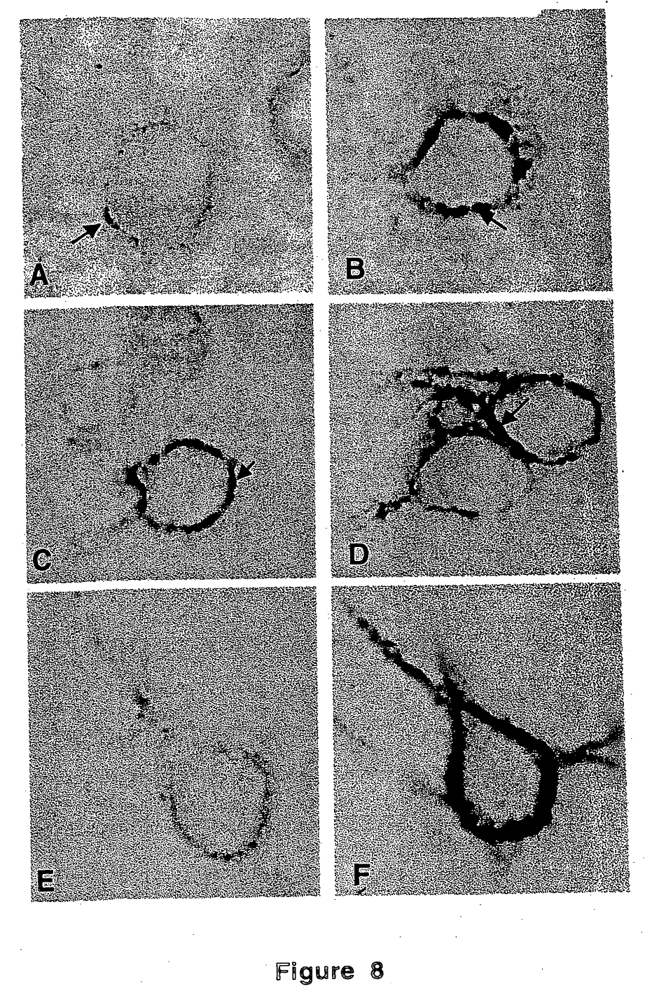

Using mice with segmental trisomy 16 (Ts65Dn), an established in vivo model of human “translocation” DS (Holtzman et al., Proc. Natl. Acad. Sci. USA 93:13333-13338, 1996), we found that, at two months of age and prior to the appearance of any neuropathological alterations, many neurons in the neocortex and basal forebrain contained enlarged early endosomes similar to those seen in AD and DS brain (FIG. 8). At six months of age, the endosomal alterations were present within the majority of neurons in the neocortex and basal forebrain and in a substantial number of hippocampal neurons. The abnormal activity of the EP in the Ts65Dn animals is similar to that seen in human SAD and DS.

To further evaluate the use of Ts65Dn mice as an in vivo model of AD-like endocytic alterations to AD, we have examined brain tissue from Ts65Dn and 2N mice using these antibodies to rab7 and LBPA. We found that prior to the appearance of detectable neurodegenerative changes, enlarged rab7- and LBPA-positive late endosomes were seen in the majority of neurons in the basal forebrain and neocortex of Ts65Dn mice, which were not seen in age-matched, 2N littermate controls

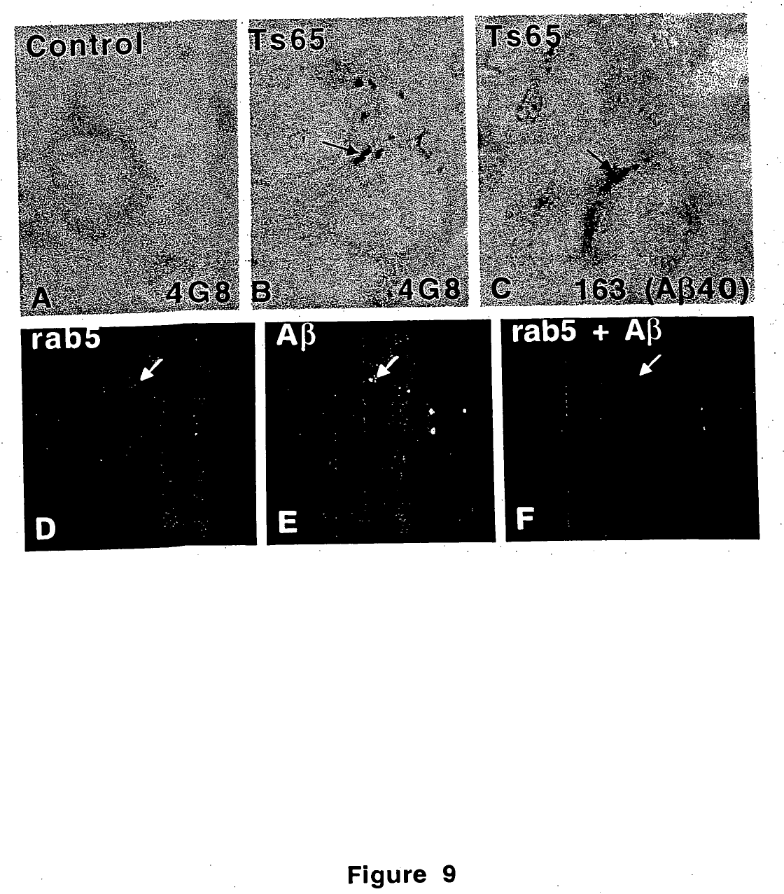

We also found in Ts65Dn mice increased neuronal content of APP and the presence of intraneuronal Aβ within vesicular compartments (FIG. 9A). An antibody directed to the 17-24 region of the Aβ peptide was used to probe brain tissue from the Ts65Dn mouse (FIG. 9B), as well as postmortem brain tissue from the neocortex of cases of human fetal, infant, and young DS, and cases obtained from adult patients in the very early stages of AD.

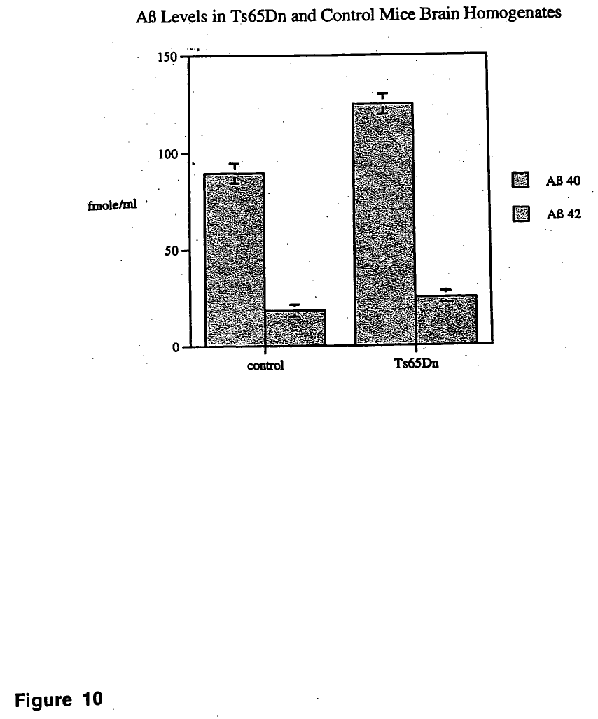

Using immunocytochemical techniques and light or confocal microscopy, we found the presence of small and discrete deposits of anti-Aβ immunoreactive material within neurons of the Ts65Dn mice (FIG. 9D-F), the early stage DS cases, and the cases in the earliest stages of AD. None of the Ts65DN mice, the early stage DS cases, or the early stage AD cases displayed evidence of extracellular β-amyloid protein in those same brain regions where intracellular Aβ was detected. Additionally, we have seen an increase in Aβ detected by sandwich ELISA in homogwnates prepared from Ts65Dn mouse brain vs. control mouse brain (FIG. 10). We have found both Aβ40 and Aβ42 to be increased by approximately 1.4 times.

In the cingulate cortex, hippocampus, basal ganglia, temporal cortex, and brain stem, both Aβ40 and Aβ42 levels were greater in the Ts65Dn mice. In all regions examined, Aβ340 levels were at least 2-fold greater in the Ts65Dn mice, a larger increase than would be predicted based solely upon the triplication of the App gene leading to 1.5-fold more APP expression. Additionally, we have detected by immunolabeling intracellular Aβ in neurons from Ts65Dn mice, whereas neurons from 2N mice fail to label with multiple anti-Aβ antibodies. Much of the intracellular Aβ in Ts65Dn neurons colocalized with abnormally large early endosomes, as we have also found in neurons from early-stage human AD cases. Western blot analysis, using a portion of the same brain-region homogenate analyzed by ELISA, showed an increase in APP levels and a disproportionate increase in CTF levels in the Ts65Dn mice compared to the 2N mice. These findings are consistent with altered endocytic pathway function in AD and Ts2 1 leading to increased β-cleavage of APP and increased Aβ production.

Other similarities among the Ts65Dn mice, early DS, and early stage AD were observed. These included abnormalities in proteins associated with the regulation of the activity of the EP.

The Ts65Dn mouse can serve as a model for early stages of cellular pathology and Aβ formation seen in sporadic AD and DS that are not modeled by existing transgenic mouse models based on mutations associated with familial forms of AD in the presenilin genes and APP gene. This model is useful for assaying candidate compounds for their ability to decrease the foregoing abnormalities and to decrease intracellular production of Aβ. Additionally, standard mouse learning and behavior paradigms known to those skilled in the art can be used to assess brain function in Ts65Dn mice administered candidate compounds.

The Ts65Dn mouse provides other uses as an in vivo model of the EP upregulation seen in AD and DS. For example, by examining the other mice that are only partially trisomic for the human homolog of chromosome 21, we can gather additional information on the specificity of the endosomal system upregulation and begin to dissect the genes that may play an essential role. Crossing Ts65Dn mice with a mouse carrying a balanced translocation of chromosome 16, T(12;16)1Cje (Huang et al., Arch. Biochem. Biophys. 344:424-432, 1997) generates four genotypes: litter-matched normal control mice; Ts65Dn;

the previously described segmental trisomy Ts1Cje (Sago et al., Proc. Natl. Acad. Sci. USA 95:6256-6261, 1998); and the poorly characterized segmental trisomy Ms1Ts65. The T(12;16)1Cje mice are phenotypically normal because the translocation is balanced. The Ts1Cje mouse is trisomic for the region of mouse chromosome 16 from the Sod1 to Mx1 genes, although the Sod1 gene is inactive (Sago et al., Proc. Natl. Acad. Sci. USA 95:6256-6261, 1998). These mice do not have an extra copy of the App gene. Ts1Cje mice have less severe learning deficits than do the Ts65Dn mice and, unlike the Ts65Dn mice, do not show degeneration of basal forebrain cholinergic neurons (BFCN) (Sago et al., Proc. Natl. Acad. Sci. USA 95:6256-6261, 1998). The Ms1Ts65 mice are trisomic for the region of mouse chromosome 16 from the App gene to, but not including, the Sod1 gene. Developmental retardation and BFCN degeneration have not been characterized in the Ms1Ts65 mouse.

Recent studies indicate pervasive early endosome abnormalities in Ts65Dn mice, and that App gene dosage appears to modulate this phenotype. Specifically, vulnerable hippocampal, neocortical, and CBF neurons in Ts65Dn mice develop enlarged early endosomes, and increased immunoreactivity for markers of endosome fusion (e.g., rab5, EEA1, and rabaptin5), and endosome recycling (e.g., rab4) similar to observations in AD and Ts2l brains. Selectively deleting one copy of App or a small portion of the MMU16 segment containing App from Ts65Dn mice eliminated the endosomal phenotype. This was performed by comparing brain tissue from Ts65Dn mice with the Ts1Cje mouse and Ts65Dn/App+/+/−, a mouse line generated by crossing Ts65Dn with an App knockout mouse, essentially creating a segmental trisomy mouse with two copies of the App gene. Ts65Dn/App+/+/−mice lack a behavioral and morphologic phenotype of Ts65Dn mice. Moreover, overexpressing App at high levels in mice did not alter early endosomes, implying that one or more additional genes on the triplicated segment of MMU16 are also required for the Ts65Dn endosomal phenotype. In summary, these results identify an essential role for App gene triplication in endosomal abnormalities found in AD and Ts2 1.

While the App gene in this region is likely to be of importance, we suspect that a dosage-effect for APP will not fully explain the Ts65Dn endosomal system upregulation, as we have not seen abnormally large endosomes in the APPswe transgenic mice nor do APP overexpressing mice develop BFCN degeneration. Characterizing the Ms1Ts65 mice that carry an extra copy of App but lack many of the other genes over represented in Ts65Dn mice will directly complement analysis of the other mice.

It is highly likely that crossing the Ts65Dn mice with mice expressing human APOE ε4 will enhance EP upregulation, similar to the greater neuronal EP upregulation seen in AD cases with the APOE ε4 genotype. This may be an even better model of EP abnormalities in which to study AP production and Aβ deposition, as well as to test candidate drugs for efficacy and safety.

We further predict that altering lysosomal function will create a better model of AD pathology, a model that will include more extensive neurodegeneration and allow us to establish a relationship between upregulation of the EP and lysosomes and neuronal atrophy. We can modulate lysosomal function by inhibiting lysosomal proteases in vivo individually or by enzyme class in any of these in vivo mouse models.

In one method, infusion pumps are implanted in animals at 8 weeks of age and the animals are sacrificed 28 days later. We can perform the following assessments: (a) determine cytochemically and biochemically whether administration of a candidate compound prevents or reduces the EP activity abnormality; (b) assess neurodegeneration in the inhibitor treated mice in the presence and absence of candidate compounds; and (c) assess any changes in β-amyloid productiondeposition.

To induce lysosomal dysfunction in vivo, the activity and/or expression of a specific cathepsin or groups of cathepsins is modified by chronic intraventricular infusion of appropriate inhibitors. In one example, proteases are inhibited in vivo in the mouse brain using an ALZET osmotic pump brain infusion kit in conjunction with model 2004 mini-osmotic pumps (0.25 mL/hr delivery rate, 28 days) (Frautschy et al., J. Neurosci. 18:8311-8321, 1998). The following inhibitors (with approximate dosages) are suitable for inducing lysosomal dysfunction: leupeptin (0.5 mmol to 4 mmol) is a broad-spectrum inhibitor of thiol proteases (e.g., Cat B, Cat L, Cat S); pepstatin (0.1 mmol to 1 mmol) inhibits Cat D, and inhibits mouse aspartyl proteases systemically at these concentrations; and Z-PAD (10 mmol to 100 mmol) inhibits thiol proteases but with greater specificity than leupeptin for Cat B and Cat L. Aprotinin (1 mmol to 30 mmol) broadly inhibits serine proteases and is principally used as a control since the major proteases in lysosomes are not of the serine type.(Green and Shaw, J. Biol. Chem. 256:1923-1928, 1981; Ohnishi et al., Cancer Res. 50:1107-1112, 1990; Umezawa, H. Academic Press, Vol. XLV, Part B, 1976; Frautschy et al., J. Neurosci. 18:8311-8321, 1998; Ivy et al., Adv. Exp. Med. Biol. 266:31-45, 1989; Ivy et al., Brain Res. 498:360-365, 1989; Kuki et al., Dementia 7:233-238, 1996; Okada et al., Neurosci. Res., 19:59-66, 1994; Bednarski et al., Exp. Neurol. 150:128-135,1998; Bednarski et al., J. Neurochem. 67:1846-1855, 1996; Bednarski et al., J. Neurosci. 17:4006-4021, 1997; Van Noorden et al., J. Rheumatol. 15:1525-1535, 1988). Doses were chosen at an anticipated effective dose and a second, higher dose. Other inhibitors are also useful for inducing lysosomal dysfunction, including, for example, vinyl sulfone inhibitors that are selective for cysteine cathepsins and have poor affinity for calpains, and peptidyl fluoromethylketone inhibitors that preferentially inhibit Cat B (Palmer et al., J. Med. Chem. 38:3193-3196, 1995; Ahmed et al., Biochem. Pharmacol. 44:1201-1207, 1992; Esser et al., Arthritis Rheum 37:236-247, 1994; Riese et al., J. Clin. Invest. 101:2351-2363, 1998).

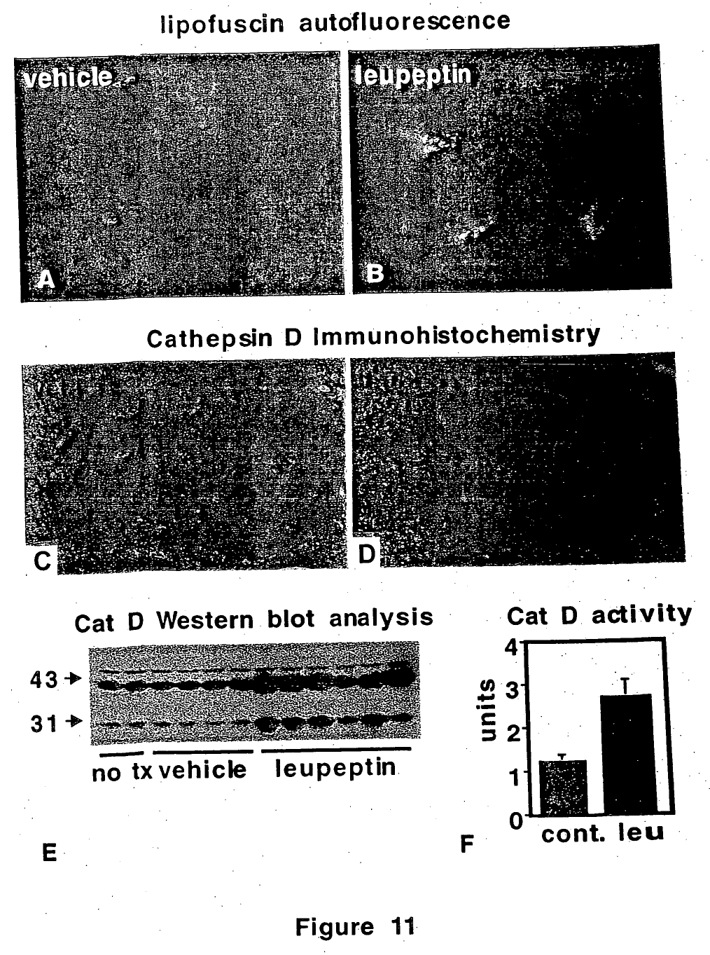

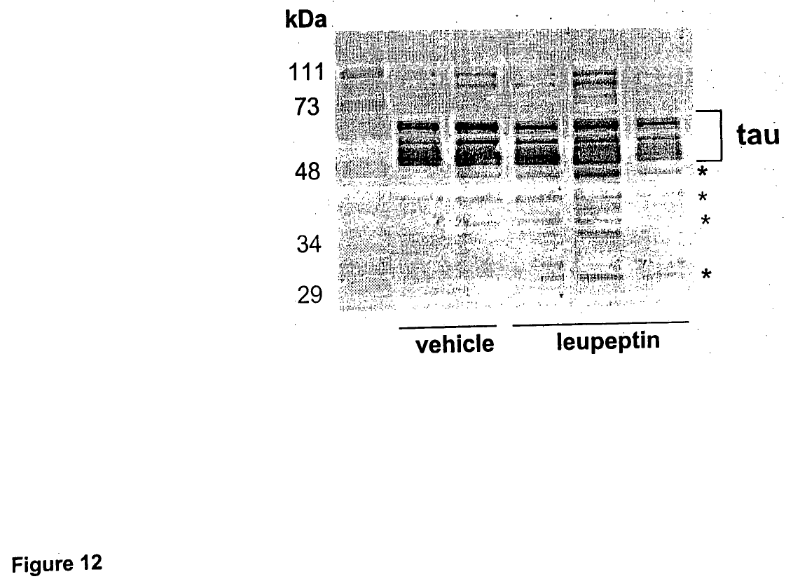

To administer the candidate compounds in the presence or absence of inhibitor(s), eight week old mice are anesthetized with pentobarbital (40-50 mg/kg i.p.) and placed in a stereotaxic apparatus with mouse adapter (David Kopf Instruments, Tujunga, Calif.). After sacrifice, placement of the probe into the lateral ventricle is confirmed and one hemi-brain immersion-fixed and the other hemi-brain homogenized for protease assays. We determine the efficacy of the candidate compound by assaying the activity of proteases in brain homogenates (e.g., Cat B, Cat L, Cat D, and trypsin (Brunk et al., Free Radic. Biol. Med. 19:813-822, 1995; Marotta and Nixon, J. Neurochem. 43:507-516, 1984; Koshikawa et al., Am. J. Pathol. 153:937-944, 1998); see, for example, Cat D shown in FIG. 11F). Chronic inhibition (7+days) by chloroquine or leupeptin infusion results in accumulation of lipofuscin-like material in neurons while aprotinin infusion, which does not inhibit any major lysosomal protease, does not (Arai et al., Ann. Neurol. 38:649-652, 1995; Ivy et al., Adv. Exp. Med. Biol. 266: 31-45, 1989; Ivy et al., Brain Res. 498:360-365, 1989; Kuki et al., Dementia 7:233-238, 1996) (compare autofluorescence in neurons from a leupeptin treated mouse (FIG. 11B) with a vehicle treated (20 mM HEPES) mouse (FIG. 11A). Lipofuscin accumulation in neurons from inhibitor treated mice is assessed by two criteria: autofluorescence emission (FIG. 11B) and EM morphological appearance. Lipofuscin granules are defined at the EM level as having a single layer of membrane and a bipartite structure consisting of electron lucent lipoid material and electron dense material (Cataldo and Nixon, Ann. N.Y. Acad. Sci. 679: 87-109, 1993).

Dysfunction of a particular protease or group of proteases may lead to a compensatory increase in levels of other lysosomal proteases (Bednarski and Lynch, Neuroreport 9:2089-2094, 1998). Analysis of the LS is performed, using assays described herein, to determine the impact of inhibitor infusion on the overall expression of most lysosomal hydrolases, hydrolases inhibited by one or more of the treatments (Cat D, Cat B), and a probe that is not affected by any of the inhibitors (LAP). In FIGS. 11C-F, increased brain levels of Cat D, a protease not directly affected by leupeptin, is shown by immunohistochemistry (FIG. 11D), Western blot analysis (FIG. 11E), and activity assay (FIG. 11F) following 4 weeks of intraventricular infusion of leupeptin. Increases in Cat D are particularly relevant to models of AD because Cat D activation has been shown to lead to activation of some cell death pathways and because Cat D itself has recently been identified as a potential β-secretase (Grüninger-Leitch et al., Nature Biotechnol. 18: 66-70, 2000).

Several studies have recently raised the possibility that abnormal forms of tau are metabolized in lysosomes. Western blot analysis of the cytoskeletal associated protein tau in brain revealed in increase in lower molecular weight forms of the protein (Mr˜30-34 kDa) in leupeptin-infused mice, suggesting an important lysosomal component to tau processing in neurons (FIG. 12). There are numerous assays for tau known in the art. For example, the processing of tau by immunocytochemistry and western blot analysis is performed using an antibody that recognizes both phosphorylated and non-phosphorylated forms (Adamec et al., Brain Res. 757:93-101, 1997). Abnormal tau conformation and hyperphosphorylated tau is examined using suitable monoclonal antibodies (e.g., MC1 (Jicha et al., J. Neurosci. Res. 48:128-132) and AT8 (Innogenetics, Gent, Belgium)). EM analysis can be performed to characterize cytoskeletal changes in neurons (Nixon, Bioessays 20:798-807, 1998). Alternatively, the magnitude of neurodegeneration in the mice can be determined by Nissl stain.

In addition, Aβ levels can be determined in mice administered inhibitors with or without candidate compounds. Levels of APP, APPs, APP carboxy-terminal fragments, and Aβ production can be examined using methods described herein. Additionally, the presence of AP in endosomal and lysosomal compartments is assayed by double-label immunocytochemistry using antibodies to Aβ and appropriate markers for these compartments (e.g., rab5).

### EXAMPLE 7

### Ts2l Models of AD-Like Early Endosomal Enlargement

We have used primary fibroblasts obtained from Ts21 individuals to explore these cells as an in vitro model of AD-related endocytic alterations. Using antibodies against early endosomes (rab5 and EEA1), we have found that Ts2l fibroblasts display early endosomal enlargement resembling that seen in neurons of AD and Ts21 brain. In contrast to the Ts21 fibroblasts, control 2N fibroblasts were found to have smaller, uniformly sized early endosomes. Double-labeling using both rab5 and EEA1 antibodies showed that these proteins colocalized within the same compartment. Rab4, which labels recycling endosomes, showed a qualitative increase in the number of immunoreactive vesicles in the Ts2 1 cells compared to 2N. Western blot analyses of cell lysates showed increased levels of rab4 in Ts2l when compared to 2N fibroblasts. These morphologic endocytic alterations in Ts21 fibroblasts (i) suggest increased early endosomal uptake, fusion, and recycling; (ii) confirm the Ts21 genetic predisposition to endocytic alterations; and (iii) validate these cells as an in vitro model with which to experimentally examine the cause of these early endocytic perturbations and their consequences.

When we compared Ts2 1 to 2N fibroblasts, LBPA immunoreactivity was associated with swollen vesicular compartments. Double-label immunofluorescence with rab7 and LBPA showed rab7 colocalization with the majority of enlarged LBPA-positive late endosomes. These results demonstrate that late endocytic abnormalities occur in Ts21 fibroblasts and that these changes morphologically resemble those in AD and Ts21 brain.

We sought evidence of increased endocytosis in Ts21 fibroblasts by functional assays using transferrin and HRP as indices of receptor-mediated and fluid-phase internalization. Following labeling on ice and a short incubation at 37° C., Ts2 1 fibroblasts showed an increase in the labeling of transferrin-positive endocytic vesicles compared to 2N fibroblasts, suggesting increased receptor-mediated uptake. Following fluid-phase loading of HRP into early endosomes, an ultrastructural examination showed an increase in the number and size of electron dense HRP-positive vesicular compartments in the Ts21 fibroblasts compared to 2N. In the Ts2 1 fibroblasts, many compartments containing HRP tracer appeared to be undergoing homotypic and heterotypic fusion and morphologically were consistent with early and recycling endosomes or early to late endocytic transfer intermediates such as multivesicular bodies. These finding suggest that the morphologic changes in early endocytic compartments seen in AD and Ts21 are the consequence of altered endocytic function.

### EXAMPLE 8

### Endocytic Abnormalities in Ts65Dn Mice and Ts21 Fibroblasts are APP Expression-Level Dependent

When we crossed Ts65Dn mice to an App knockout mouse, reducing App copy number to diploid but leaving the rest of the Ts65Dn trisomic region intact, normal early endosomal morphology was restored (Cataldo et al., J. Neurosci. 23: 6788-6792, 2003). We now have employed siRNA to knockdown APP expression in Ts2l fibroblasts, which show an increase in APP levels relative to their 2N counterparts. Sister cultures exposed to App siRNA (100 nM) display an approximate 50-75% reduction in APP expression, roughly approximating 2N levels. The App siRNA sequence used to significantly knock down App synthesis was 5′-AACATGCACATGAATGTCCAG-3′. Although EEA1 expression levels were similar in control and App siRNA Ts21 fibroblast cultures, reversal of the enlarged endosome phenotype was evident, supporting the concept of the importance of App gene dosage in the endosome enlargement phenotype in Ts21 and in vivo and in vitro models of Ts21. Moreover, rab4 levels were knocked down in App siRNA cultures relative to no treatment (in both 2N and Ts21 fibroblasts) and mock transfected cultures.

As in the Ts65Dn mouse, reducing APP expression reduced the number of enlarged EEA1-positive early endosomes; following APP knockdown, EEA1 immunoreactivity in Ts21 cells was found associated with small vesicles and diffusely throughout the cytosol, suggesting that APP knockdown may result in a hyponormal endosomal phenotype. By western blot analysis, EEA1 protein levels were found to be slightly higher in the Ts21 compared to 2N fibroblasts and reduced by the APP knockdown. Rab4 and to a greater extent rab7 protein levels were also increased in the Ts21 fibroblasts. The levels of both rab4 and rab7 were reduced following APP knockdown, rab4 to below 2N levels. These biochemical findings are in agreement with our immunolabeling studies of Ts21 fibroblasts showing alterations in early and late endocytic compartments and confirms that APP expression levels are a key modulator of the endocytic alterations in the Ts21-background.

### EXAMPLE 9

### Evaluation and Knockdown of Additional Trisomic Genes

App overexpression alone has no detectable effects on endosomes, implying that App triplication is necessary, but not sufficient, to cause AD-related endosome dysfunction and that one or more additional genes within the Ts65Dn trisomic segment of MMU16 is required. Our observations that fibroblasts from Ts21 subjects exhibit endosomal pathology similar to that seen in neurons in AD and Ts21 brains, which can be reversed by lowering App expression, presents an unique opportunity to identify genes, in addition to App, that are required for the endosomal phenotype and, therefore, represent exceptional candidates to target to AD.

To identify these genes, Ts21 or Ts65DN primary skin fibroblasts and 2N control fibroblasts are maintained at 37° C. and 5% CO₂ in MEM (Invitrogen) supplemented with 15% fetal bovine serum (Hyclone), 2 mM glutamine (Invitrogen), and penicillin/streptomycin (Invitrogen). Fibroblasts (approximately 1 million cells) used for transfection studies are seeded onto poly-lysine (Sigma) coated 100 mm culture dishes. DNA templates of targeted siRNA sites for trisomic genes are custom designed using proprietary software (Qiagen). siRNAs with regions of 14-15 contiguous bases of sequence similarity between the siRNA and an mRNA effectively induce silencing. siRNA synthesis is performed using a Silencer siRNA Construction Kit (Ambion) per the manufacturer's instructions. Transfection of cultured cells is performed 24 hr after plating using siPORT lipid transfection agent (Ambion) according to protocol. Initial screens are performed in duplicate for each of three test siRNA constructs individually at final concentrations of 100 nM and as a cocktail of all three siRNAs at 50 nM each to lower stringency for positive hits. This will permit us to measure reductions in protein expression directly to assess efficacy of knockdown. The indices of endosome phenotype reversal include EEA1 redistribution and reduction of EEA1-positive early endosomes and rab7-positive late endosomes assessed by immunocytochemistry and confirmation of these results by quantification of transferrin uptake, a measure of endocytic uptake, and normalization of rab4 and rab7 measured by immunoblot analysis. Following siRNA silencing, cells attached to the coverslip are fixed with 4% formaldehyde and immunolabeled with an anti-EEA1 antibody and separately with an anti-rab7 antibody. Cells are permeabilized with 0.1% saponin and labeled with Alexa Fluor 568 and/or Alexa Fluor 488 conjugated secondary antibodies (Molecular Probes). Coverlips are mounted in gelmount (Biomeda) and examined by laser confocal microscopy (Leica). In our preliminary experiments using App siRNA, EEA1 (and rab7) immunolabeling readily detected a reversal of endosomal phenotype in Ts2 1 or Ts65DN fibroblasts. Additionally, EEA1 protein levels were similar in 2N fibroblasts and Ts21 fibroblasts, and did not change in response to App RNA silencing. Thus, changes in endosome size and the endosome-to-cytosol redistribution of EEA1 associated with phenotype reversal by gene silencing are unlikely to be confounded by alterations in EEA1 levels. For an siRNA to pass this initial screen, more than 50% of the cells should display reversal by immunocytochemistry (EEA1 redistribution and endosome size reduction to that in diploid cells) and/or the transferrin uptake assay (each test should track the other and be mutually confirmatory). A comparison of these parameters between 2N fibroblasts and s21 or Ts65Dn fibroblasts treated with App siRNA serves as a positive control condition in all experiments.

There are at least 77 genes present in three copies in Ts65Dn mice: Mrp139, Jam2, Atp5a, Gabpa, App, Adamts5, Znf294, C21orf6, Usp16, Cct8, C21orf7, Bach1, Cldn17, Cldn8, Krtap11-1, Tiam1, Sod1, Ctdbp, Hunk, C21orf108, C21orf63, C21orf59, Synj1, C21orf66, Olig2, Ifnar2, Il10rb, Ifnar1, Ifngr2, C21orf4, Gart, Son, Donson, Cryzl1, Itsn1, Atp5o, Slc5a3, Mrps6, Kcne2, C21orf51, Kcne1, Dscr1, Clic6, Runx1, C21orf18, Cbr1, Cbr3, C21orf5, Kiaa0136, Chaf1b, Sim2, Hlcs, Dscr5, Ttc3, Dscr3, Dyrk1a, Kcnj6, Kcnj15, Erg, Ets2, Dscr2, Wdr9, Hmgnl, Wrb, Sh3bgr, B3galtS, Igsf5, Pcp4, Dscam, Bace2, Fam3b, Mx2, Mx1, Tmprss2, Ankrd3, C21orf25, and Znf295.

Genes that are present in three copies in Ts2 1 individuals and are good candidates for contributing to the observed endosomal phenotype are listed in Table 1.

TABLE 1

| Notation | Name and function |
|---|---|
| BACE2 | aspartyl protease ASP1 |
| SOD1 | Cu/Zn superoxide dismutase |
| PCBP3 | poly (rC)-binding protein 3 |
| KCNJ6 | Inward rectifier potassium channel 2 (hiGIRK2) |
| ETS2 | Erythroblastosis virus oncogene homolog 2 |
| SIM2 | transcription factor SIM2 |
| DYRK1A | Dual-specificity tyrosine-Y-phosphorylation regulated |
| | kinase |
| DSCR1 | Down syndrome proline-rich protein |
| MRPS6 | Mitochondrial ribosomal protein S6 |
| CBR1 | Carbonyl reductase (NADPH) 1 |
| DSCAM | Down syndrome cell adhesion molecule |
| SYNJ1 | Synaptojanin-1 |
| TIAM1 | T-lymphoma invasion and metastasis inducing TIAM1 |
| | protein |
| GRIK1 | Glutamate receptor (GluR5) |
| C21orf5 | mRNA for KIAA0933 protein |
| KIAA0184 | mRNA for KIAA0184 protein |
| PCNT2 | Pericentrin, kendrin (KIAA0402) |
| PDXK | Pyridoxal kinase |
| PFKL | Liver-type 1 - phosphofructokinase |
| PCP4 | Brain specific polypeptide PEP19 |
| ADAMTS5 | aggrecanase-2 mRNA |
| ADAMTS1 | metalloproteinase type 1 motifs |
| ZNF294 | mRNA for KIAA0714 protein |
| PSMD4 | proteasome 26S subunit gene |
| TMPRSS2 | transmembrane protease, serine 2 |

### EXAMPLE 10

### Models of AD Neuropathology Following Leupeptin Infusion

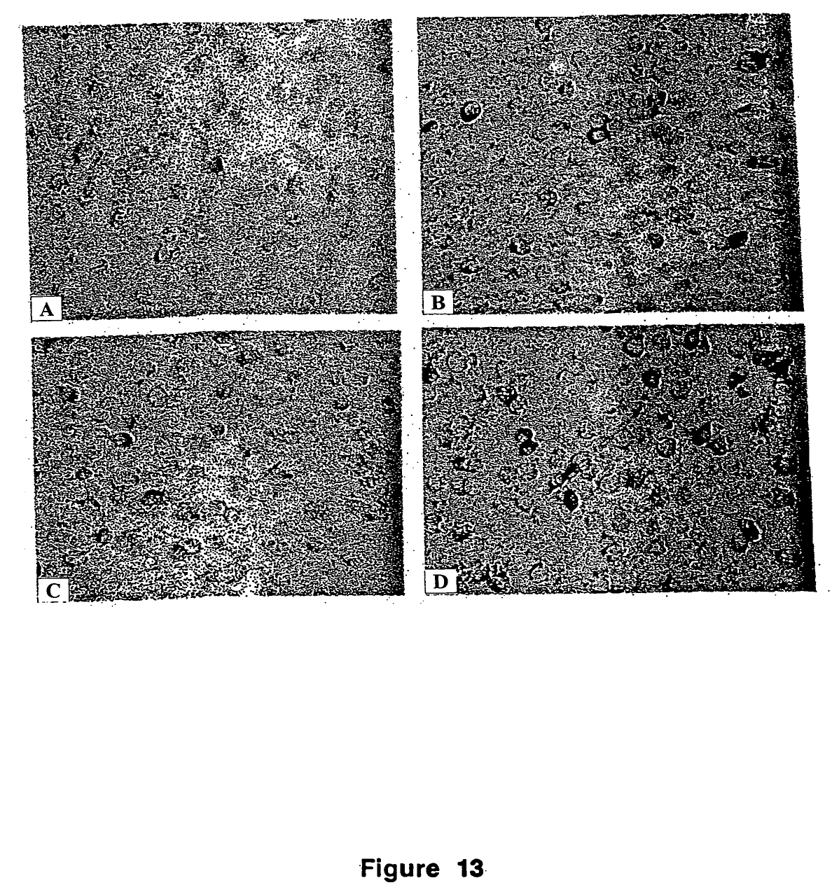

Ts65Dn and control mice were infused intraventricularly with leupeptin (5 mg/ml) for 4 weeks. As previously shown in FIGS. 11C and 11D, leupeptin infusion resulted in an increased level of Cat D detected by immunolabeling in a 2N control mouse (FIG. 13B) when compared to a 2N control mouse infused with vehicle alone (FIG. 13A). The degree of lysosomal system activation, however, was found to be greater in a Ts65Dn mouse infused with leupeptin (FIG. 13D) compared with a Ts65Dn mouse infused with vehicle alone (FIG. 13C). This enhanced lysosomal system activation seen in the Ts65Dn mouse more closely mimics the lysosomal system activation seen in human AD, and is likely to make this a valuable model in which to examine the relationship between EP abnormalities, lysosomal system dysfunction, and cell death.

As a second example of the usefulness of inducing lysosomal system dysfunction by protease inhibitor infusion, we treated PS1_{M146L}/APPswe transgenic mice, an established model of AD-like pathology (Duff et al., Nature 383:710-713, 1996). Infusion pumps containing 5 mg/mL leupeptin were implanted into PS1_{M146}L/APPswe transgenic mice at 1 year of age. Animals were sacrificed 4 weeks later.

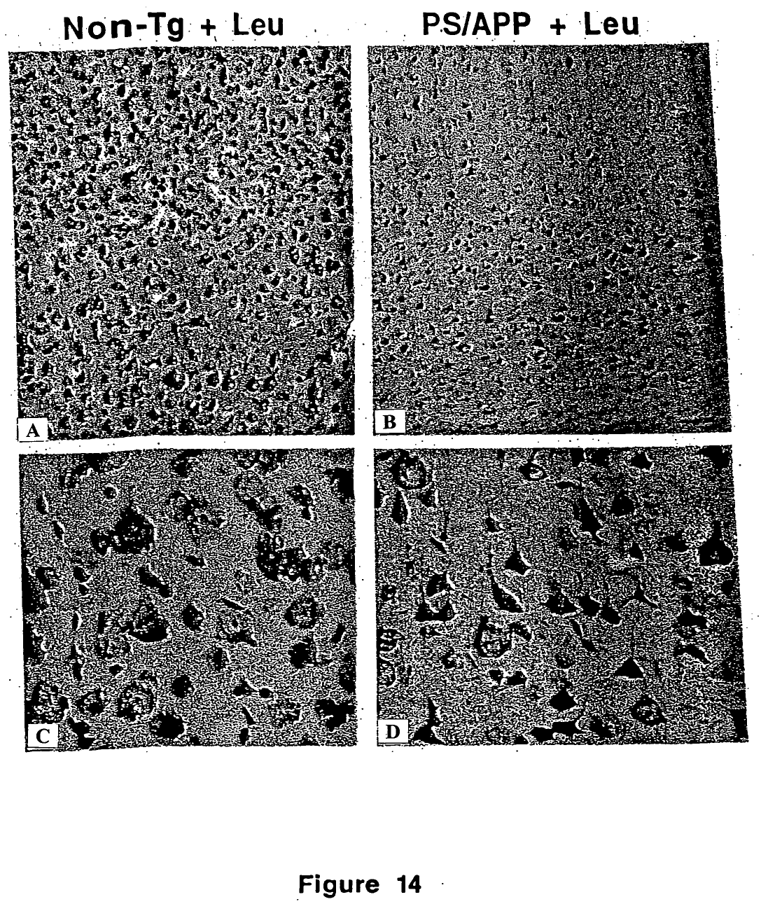

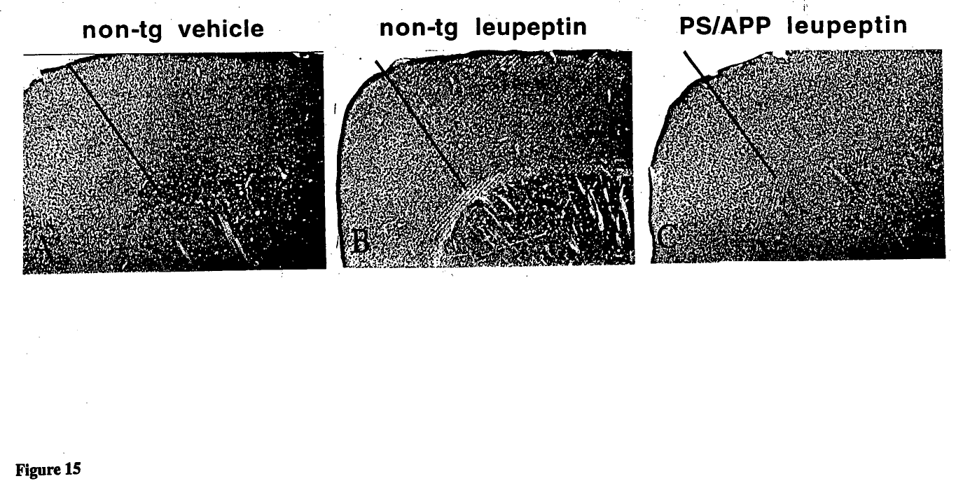

Nissl staining of a PS_{M146}L/APPswe mouse infused with leupeptin showed substantial cortical neuronal hypotrophy, chromatolysis and possibly loss of neurons (FIGS. 14B and 14D), when compared to a non-transgenic littermate also infused with leupeptin (FIGS. 14A and 14C). We confirmed this observation by measuring the thickness of the cortical mantle in these mice (FIG. 15). While leupeptin treatment resulted in a small decrease in thickness of the cortical mantle of a non-transgenic mouse (compare FIGS. 15A and 15B), leupeptin treatment of a PS1_{M146}L/APPswe mouse reduced the thickness of the cortex by over 20%. One current limitation of the existing mouse models of AD is their failure to show substantial neuronal atrophy and/or neuronal cell loss, key features of the disease in humans. Our results strongly suggest that disruption of the lysosomal system will enhance existing animal models of AD pathology by generating a neuronal phenotype much closer to that of the human tissue.

### EXAMPLE 11

### Leupeptin Infusion Increases Amyloid Deposition and Aβ Levels

Infusion pumps and brain cannulae containing 5 mg/mL leupeptin were implanted into mice. Following four weeks of leupeptin treatment, mice were sacrificed and fixed tissue examined for lipofuscin autofluorescence, an indication of reduced lysosomal hydrolysis and accumulation of nondegraded material. There was an increase in lipofuscin autofluorescence in leupeptin-treated mice (FIG. 9B) when compared to animals receiving vehicle (20 mM HEPES) alone (FIG. 9A). Additionally, Cat D, a protease not directly affected by leupeptin, was examined in these mice. Immunolabeling of tissue sections with antibodies specific for Cat D showed a substantial increase in Cat D expression within neurons of leupeptin-treated animals (FIGS. 9C and 9D). Western blot analysis of the contralateral hemisphere to cannula placement confirmed this finding. Expression of both the mature form and heavy chain of Cat D was found to be increased (FIG. 9E). Finally, Cat D activity was measured in these same brains and found to be increased 3-fold in the leupeptin-treated mice over mice that received vehicle alone (FIG. 9F). Similar increases in Cat B immunolabeling and expression by western blot analysis have been seen in leupeptin-treated mice, as has an increase in lysosomal acid phosphatase activity.

We also performed protease inhibitor infusion experiments in PS1_{M146}L/APPswe transgenic mice. Infusion pumps containing either 2 mg/mL pepstatin or 10 mg/mL leupeptin were implanted into PS1_{M146}L/APPswe transgenic mice at 8 weeks of age. Animals were sacrificed at 12 weeks of age, and total Aβ levels were determined by ELISA following formic acid extraction (FIG. 10A). Total Aβ levels in the pepstatin-treated mice were found to be reduced to half the level detected in untreated 12 week old PS1_{M146}L/APPswe transgenic mice. Leupeptin treatment, on the other hand, increased total Aβ by 1.8 times when compared to untreated mice. We have additionally measured the activity of Cat D in the brains of untreated, pepstatin-treated, or leupeptin-treated mice (FIG. 10B). As we predicted, infusion of the aspartyl-protease inhibitor pepstatin reduced activity of Cat D in the brain. Treatment with leupeptin, a cysteine protease inhibitor, increased Cat D activity, consisent with the known role of cysteine cathepsins in degrading Cat D in the lysosome.

Additionally, western blot analysis of the cytoskeletal associated protein tau in brain revealed an increase in lower molecular weight forms of the protein (Mr ˜30-34 kDa) in leupeptin-infused mice, suggesting an important lysosomal component to tau processing in neurons (FIG. 11).

Assays

Using transfected cells and in vivo models described herein, candidate compounds can be assayed for their ability to reduce the alterations in endocytosis and hydrolase trafficking to early endosomes observed in human brain. Additionally, candidate compounds can be assayed for their ability to decrease Aβ production. These assays are described in more detail below.

Measurements of Endocytosis

Early endosomal volumes and identities of modulator proteins associated with increased endocytosis are determined by immunolabeling (e.g., by Western blot analysis) with rab5, rabaptin 5, or EEA 1, using standard techniques.

Fluid-phase endocytosis rates are determined by incubating living cells in 5 mg/ml of dextran in DMEM for various time periods (5 min to 2 hr). Rates of uptake are determined visually using fluorescent-dextran or, alternatively, quantitatively using biotin-dextran coupled to a colorometric assay (Prchla et al., J. Cell Biol. 131:111-123, 1995).

Receptor mediated uptake is assayed by binding fluorescent-labeled transferrin (Cy3-transferrin, 25 μg/ml) at 4° C. for 1 hr to living cells followed by warming to 37° C. for periods of 0, 3, 6, 8, and 20 min. Cells fixed after 6 min, which we have found to have maximum uptake into early endosomes, are quantitated for the number of Cy3-positive endosomes. In addition, double labeling experiments are performed to show the coincidence of Cy3-transferrin and rab5. In these assays, it is preferred that transformed cells are compared to parental cells.

Localization of Lysosomal Hydrolases to Early Endosomes

Double-labeling with the specific antibodies (rab5 and Cat D or Cat B) and confocal microscopy are used to show coincidence of lysosomal hydrolases and early endosomes. Uptake of BODIPY-pepstatin, which specifically binds to enzymatically active Cat D, is a useful method to detect increased Cat D levels in early endosomes. 1 mM BODIPY-pepstatin is added to the growth medium, and cells washed at various time-points (10 min to 5 hr), fixed, and labeled with antibodies specific for early endosomes and examined as above. The specificity of increased BODIPY-pepstatin uptake is determined by including an excess of unlabeled pepstatin in the medium (20 mM). Cat D and Cat B are immunoprecipitated from cell lysates and from the growth medium using specific antibodies. In parallel cultures, 5 mM Man-6-P is added to the chase medium to promote secretion of hydrolases delivered to the cell surface/early endosomes into the medium (Watanabe et al., Proc. Natl. Acad. Sci. USA 87:8036-8040, 1990).

A mouse from a transgenic line that is administered a candidate compound is analyzed to determine whether it exhibits evidence of decreased endocytosis or less mistrafficking of hydrolases to early endosomes compared to a transgenic mouse from the same line but not given a candidate compound. This can be performed using any of the assays described herein. In one example, sections of mouse brain are assessed by labeling with antibodies to rab5, Cat D, and MPR46. Whether endocytosis is decreased in the transgenic mice administered the compound can be determined using semiquantitative densitometry. The transgenic mice described herein can be crossed with each other, with a mouse that has increased AD-like pathology, a mouse that is expressing a polypeptide that has a mutation found in human with AD (e.g., APP, PS-1, or PS-2), or with a reporter mouse. Transgenic mice from these crosses that have been administered a candidate compound are examined, for example, at 2, 5.5, or 12 months of age.

Aβ ELISA Methods

The Aβ sandwich ELISA is generally known to those skilled in the art, with both Aβ ELISA kits (Biosource International, Camavillo, Calif.) and appropriate antibodies (e.g., 4G8, GE10; Seneteck, PLC, Napa, Calif.) available commercially. For the AP sandwich ELISA, Nunc-Immuno Plates (Nunc A/S Roskilde, Denmark) were coated overnight at 4% using antibodies specific for Aβ40 or Aβ42 in 100 mM bicarbonate buffer, pH 9.6. Remaining protein binding sites were blocked by incubating with 1% Block Ace (Yukijirushi Milk, Sapporo Japan) in PBS for 4 hours at room temperature. 10% (w/v) homogenates were prepared from a hemibrain in 20 mM Tris, 250 mM sucrose, 1 mM EDTA, 1 mM EGTA, protease inhibitors, pH 7.4, and stored frozen at −70° C. Immediately prior to being loaded on the ELISA, 1 ml of the brain homogenate was extracted in diethylamine (Sigma, St. Louis, Mich.) by adding an equal volume of 0.4% DEA in 100 mM NaCl, re-homogenized, and centrifuged for 1 hour at 100,000×g. The supernatant was collected, neutralized with 0.1 volume 0.5 M Tris, pH 6.8, and loaded in duplicate wells both neat and diluted 1:2 in EC buffer (20 mM Na phosphate, 2 mM EDTA, 400 mM NaCl, 0.2 BSA, 0.4% Block Ace, 0.95% CHAPS). The DEA extraction protocol has been shown to efficiently recover immunoreactive AP from mouse brain homogenates and leave both full-length and sAPP in the 100,000×g pellet (Savage et al., J. Neurosci. 18:1743-1752, 1998). Alternatively, conditioned media collected from cells was loaded neat and 1:2. Aβ-40 and Aβ-42 peptide standards were purchased from American Peptide Co. (Sunnyvale, Calif.), stored at −70° C., and diluted in EC buffer immediately prior to use. ELISA plates were incubated overnight at 4° C. with samples and standards. Aβ was detected by incubating for 4 hours at room temperature with an HPR-conjugated anti-Aβ antibody in 20 mM Na phosphate, 2 mM EDTA, 400 mM NaCl, 1.0% BSA. ELISA plates were developed using a color reaction (ABTS Peroxidase Substrate System, Kirkegaard & Perry, Gaithersburg, Md.) and the OD₄₅₀ read.

Isolation of Early Endosomes

Early endosomes are isolated from brain using standard techniques (e.g., the Optiprep step gradient density method (Ford et al., Anal. Biochem. 220:360-366, 1994; Graham et al., Anal. Biochem. 220:367-373, 1994; Kindberg et al., Anal. Biochem. 142:455-462, 1984)).

Cell Lines

L-cells and N2a cells are maintained at 37° C. and 5% CO₂ in high glucose DMEM supplemented with 10% FBS, 2 mM glutamax I, and penicillin/streptomycin. For selection, medium is supplemented with 400 μg/ml of G418, 200 82 g/ml of hygromycin B, and/or HAT as is appropriate. IMR-32 neuroblastoma cells are maintained in 60% DMEM, 30% Ham's-F12, 5% a MEM, 1% FCS, 4% NCS, 0.6% glucose, 200 mM glutamine, and 15 mM HEPES. Neuronal differentiation is achieved by supplementing medium with 1 mM dibutyryl cAMP and 2.5 μM 5-BDU.

Immunofluorescence Labeling

Transfected cells are incubated in 20 mM butyrate in growth medium for 24-48 hr prior to fixation to induce expression of transfected cDNA and fixed in 4% PFA, 5% sucrose in PBS (pH 7.3) at RT for 20-35 min, rinsed, and labeled with specific antibody in PBS containing 10% serum with 0.1% saponin. Coverslips are washed after primary antibody binding and secondary antibody is bound for 2 hr at RT. The coverslips are then be rinsed, mounted and examined by immunofluorescence.

Metabolic Labeling

Approximately 5×10⁵ cells are seeded in 35 mm dishes for 16 hr, followed by neuronal differentiation and/or induction of expression of transfected cDNA. Cells are incubated in methionine- and cysteine-free DMEM for 20 min before a 15-minute incubation with 100-200 μCi/mL TRANS³⁵ S-LABEL (Dupont-NEN), or for 4 hr with 500 μCi/ml for Aβ IP. Following rinses with DMEM 10% FBS and 2 mM methionine at 4° C., cells are lysed immediately or incubated with 2 mM unlabeled methionine in DMEM with 10% FBS for chase periods of 15 min to 8 hr. APP and APP metabolites are immunoprecipitated using any of a number of antibodies known to those skilled in the art, followed by separation using SDS-PAGE. Gels are exposed to X-ray film or are analyzed quantitatively using a Phosphoimager.

Immunocytochemistry and Digital Confocal Microscopic Analysis

Sections of fixed transgenic mouse brain or fixed cultured cells are processed as previously described (Cataldo et al., Neuron 14: 671-680, 1995; Cataldo et al., J. Neuropathol. Exp. Neurol. 55: 704-715, 1996; Cataldo et al., Adv. Exp. Med. Biol. 389: 271-280, 1996; Cataldo et al., J. Neurosci. 16: 186-199, 1996; Cataldo et al., Proc. Natl. Acad. Sci. USA 87: 3861-3865, 1990; Cataldo et al., Brain Res. 513: 181-192, 1990; Nixon et al., Ann. N.Y. Acad. Sci. 674: 65-88, 1992). Ts65Dn mice made transgenic for human APOE ε2 and ε4, as well as other transgenic mouse models described herein, are studied initially by immunocytochemical analysis with rab5, rabaptin 5 and EEA 1 to determine if the APOE genotype accentuates morphological changes in early endosomes. The effect of ApoE on exacerbating EP changes is further examined by crossing APOE allele-specific mice with Ts65Dn mice.

Additional Methods

A compound that decreases the activity of the EP can be further tested for AD-like abnormalities in physiology, anatomy, or behavior using assays known to those skilled in the art, including those described in U.S. Pat. No. 5,877,399, hereby incorporated by reference.

### Other Embodiments

All publications mentioned in this specification are herein incorporated by reference to the same extent as if each independent publication was specifically and individually indicated to be incorporated by reference.

While the invention has been described in connection with specific embodiments thereof, it will be understood that it is capable of further modifications. This application is intended to cover any variations, uses, or adaptations following, in general, the principles of the invention and including such departures from the present disclosure within known or customary practice within the art to which the invention pertains and may be applied to the essential features hereinbefore set forth.

What is claimed is:

1. A method for identifying a candidate compound as a compound that is useful for the treatment of Alzheimer's disease, said method comprising the steps of:
(a) providing a Tn65Dn mouse;
(b) administering a candidate compound to said mouse; and
(c) measuring the activity of the endocytic pathway, wherein a decrease in said activity, compared to the activity in a Tn65Dn mouse not contacted with said candidate compound, identifies said candidate compound as a compound that is useful for the treatment of Alzheimer's disease.

2. The method of claim 1, wherein said activity of the endosomal pathway is selected from the group consisting of endosomal fusion, endosomal recycling, expression of MPR46, accumulation of lysosomal hydrolases in early endosomes, and accumulation of AP in early endosomes.

3. A method for identifying a candidate compound as a compound that is useful for the treatment of Alzheimer's disease, said method comprising the steps of:
(a) providing a Tn65Dn mouse;
(b) administering a candidate compound to said mouse; and
(c) measuring AP formation, wherein a decrease in said Aβ formation, compared to Aβ formation in a Tn65Dn mouse not contacted with said candidate compound, identifies said candidate compound as a compound that is useful for the treatment of Alzheimer's disease.

4. A method for identifying a candidate compound as a compound that is useful for the treatment of Alzheimer's disease, said method comprising the steps of:
(a) providing a cell from a Tn65Dn mouse or a cell from a human with trisomy 21;
(b) contacting said cell with the candidate compound; and
(c) measuring the activity of the endocytic pathway, wherein a normalization in said activity, compared to the activity in a control cell not contacted with said candidate compound, identifies the candidate compound as a compound that is useful for the treatment of Alzheimer's disease.

5. The method of claim 4, wherein said activity of the endosomal pathway is selected from the group consisting of endosomal fusion, endosomal recycling, expression of MPR46, accumulation of lysosomal hydrolases in early endosomes, and accumulation of Aβ in early endosomes.

6. The method of claim 4, wherein said cell is from a cell line derived from said mouse or said human.

7. The method of claim 6, wherein said cell line is selected from the group consisting of a fibroblast cell line, a neuronal cell line, and a neuroblastoma cell line.

8. The method of claim 4, wherein said cell is selected from the group consisting of a fibroblast, a neuron, and an endothelial cell.

9. The method of claim 4, wherein said cell is in vitro.

10. A method for identifying a candidate compound as a compound that is useful for the treatment of Alzheimer's disease, said method comprising the steps of:
(a) providing a cell from a Tn65Dn mouse or a cell from a human with trisomy 21;
(b) contacting said cell with the candidate compound; and
(c) measuring Aβ formation, wherein a decrease in said Aβ formation, compared to Aβ formation in a control cell not contacted with said candidate compound, identifies the candidate compound as a compound that is useful for the treatment of Alzheimer's disease.

11. The method of claim 10, wherein said activity of the endosomal pathway is selected from the group consisting of endosomal fusion, endosomal recycling, expression of MPR46, accumulation of lysosomal hydrolases in early endosomes, and accumulation of Aβ in early endosomes.

12. The method of claim 10, wherein said cell is from a cell line derived from said mouse or said human.

13. The method of claim 12, wherein said cell line is selected from the group consisting of a fibroblast cell line, a neuronal cell line, and a neuroblastoma cell line.

14. The method of claim 10, wherein said cell is selected from the group consisting of a fibroblast, a neuron, and an endothelial cell.

15. The method of claim 10, wherein said cell is in vitro.

16. A method of identifying a gene as a gene involved in Alzheimer's disease pathology, said method comprising the steps of:
(a) providing a cell from a Tn65Dn mouse or a cell from a human with trisomy 21;
(b) contacting said cell with an agent that reduces the expression of a gene that is present in said cell in three copies; and
(c) measuring the activity of the endocytic pathway, wherein a decrease in said activity, compared to the activity in a control cell not contacted with said agent, identifies the gene as a gene that a gene involved in Alzheimer's disease pathology.

17. The method of claim 16, wherein said activity of the endosomal pathway is selected from the group consisting of endosomal fusion, endosomal recycling, expression of MPR46, accumulation of lysosomal hydrolases in early endosomes, and accumulation of AP in early endosomes.

18. The method of claim 16, wherein said cell is from a cell line derived from said mouse or said human.

19. The method of claim 18, wherein said cell line is selected from the group consisting of a fibroblast cell line, a neuronal cell line, and a neuroblastoma cell line.

20. The method of claim 16, wherein said cell is selected from the group consisting of a fibroblast, a neuron, and an endothelial cell.

21. The method of claim 16, wherein said cell is in vitro.

22. The method of claim 16, wherein said gene is selected from the group consisting of Adamts1, Adamts5, Ankrd3, Atp5a, Atp5o, B3galt5, Bace2, Bach1, C21orf108, C21orf18, C21orf25, C21orf4, C21orf5, C21orf51, C21orf59, C21orf6, C21orf63, C21orf66, C21or7, Cbr1, Cbr3, Cct8, Chaf1b, Cldn17, Cldn8, Clic6, Cryzl1, Ctdbp, Donson, Dscam, Dscr1, Dscr2, Dscr3, Dscr5, Dyrk1a, Erg, Ets2, Fam3b, Gabpa, Gart, Grik1, Hlcs, Hmgnl, Hunk, Ifnar1, Ifnar2, Ifngr2, Igsf5, Il10rb, Itsn1, Jam2, Kcne1, Kcne2, Kcnj15, Kcnj6, Kiaa0136, Kiaa0184, Krtap11-1, Mrpl39, Mrps6, Mx1, Mx2, Olig2, Pcbp3, Pcnt2, Pcp4, Pdxk, Pjkl, Psmd4, Runx1, Sh3bgr, Sim2, Slc5a3, Sod1, Son, Synj1, Tiam1, Tmprss2, Ttc3, Usp16, Wdr9, Wrb, Znf294, and Znj295.

23. The method of claim 16, wherein said agent is an siRNA that selectively reduces expression of said gene.


SEQUENCE LISTING (1 sequence)

SEQ ID NO: 1 - DNA, 21 bp, Mus musculus
aacatgcacatgaatgtccag